(19) United States
(12) Patent Application Publication
(10) Pub. No.: US 2023/0120739 A1
(43) Pub. Date: Apr. 20, 2023

(54) FLUORINE-CONTAINING ETHER COMPOUND AND METHOD OF MANUFACTURING THE SAME, COMPOUND AND METHOD OF MANUFACTURING THE SAME, FLUORINE-CONTAINING ETHER COMPOSITION, COATING LIQUID, AND ARTICLE AND METHOD OF MANUFACTURING THE SAME

(71) Applicant: AGC INC., Tokyo (JP)

(72) Inventors: Koki WATANABE, Tokyo (JP); Ryuta Takashita, Tokyo (JP)

(73) Assignee: AGC INC., Tokyo (JP)

(21) Appl. No.: 18/077,339

(22) Filed: Dec. 8, 2022

Related U.S. Application Data
(63) Continuation of application No. PCT/JP2021/021802, filed on Jun. 8, 2021.

(30) Foreign Application Priority Data
Jun. 12, 2020 (JP) 2020-102409

(51) Int. Cl. C09D 171/02; C07F 7/18; C07C 47/14; C07C 45/00; C07C 43/12; C08G 65/336

(52) U.S. Cl. CPC C09D 171/02; C07F 7/1804; C07F 7/188; C07C 47/14; C07C 45/002; C07C 43/12; C08G 65/336

(57) ABSTRACT

Provided are a fluorine-containing ether compound that can form a surface layer that excels in durability and a method of manufacturing the same, a fluorine-containing ether composition and a coating liquid, and an article having a surface layer that excels in durability and a method of manufacturing the same. A fluorine-containing ether compound is expressed by the following formula (A1) or (A2):

{R^{f}O—(R^{f1}O)ₘ₁—(R¹)ₘ₂—(CHF)ₘ₃—O—(CHF)ₘ₄}ₙ₁-Q¹(-T¹)ₙ₂ Formula (A1)

(T²)ₙ₃-Q²-(CHF)ₘ₅—O—(CHF)ₘ₆—(R²)ₘ₇—O—(R^{f2}O)ₘ₈—(R³)ₘ₉—(CHF)ₘ₁₀—O—(CHF)ₘ₁₁-Q³(-T³)ₙ₄ Formula (A2),
in the above, the symbols in the formulas are as described in the specification.

### INCORPORATION BY REFERENCE

This application is based upon and claims the benefit of priority from Japanese Patent Application 2020-102409 filed on Jun. 12, 2020, and PCT application No. PCT/JP2021/021802 filed on Jun. 8, 2021, the disclosure of which is incorporated herein in its entirety by reference.

### BACKGROUND

The present invention relates to fluorine-containing ether compounds and methods of manufacturing the same, novel compounds suitable for manufacturing the fluorine-containing ether compound or the like and methods of manufacturing such novel compounds, fluorine-containing ether compositions, coating liquids, and articles and methods of manufacturing the same.

Fluorine-containing ether compounds having a perfluoroether chain and a hydrolytic silyl group can form, on a surface of a substrate, a surface layer that is, for example, highly lubricant, water-repellent, or oil-repellent, and thus such compounds are suitably used in surface treatment agents. Surface treatment agents including fluorine-containing ether compounds are used in such cases where a property in which water repellency or oil repellency of a surface layer is less likely to decrease even the surface layer is repeatedly rubbed with fingers (friction resistance) or a property in which fingerprints that have adhered to a surface layer can be easily wiped and removed from the surface layer (fingerprint stain removability) is desired to last for an extended period of time. For example, surface treatment agents including fluorine-containing ether compounds are used as surface treatment agents for a component that constitutes a surface of a touch panel that a user touches with fingers, an eyeglass lens, or a display of a wearable terminal.

Proposed fluorine-containing ether compounds that can form, on a surface of a substrate, a surface layer that excels in friction resistance or fingerprint stain removability include a fluorine-containing ether compound having a perfluoroether chain and a hydrolytic silyl group (International Patent Publication No. WO2017/022437).

### SUMMARY

There is a demand for, for example, a surface treatment agent that can be applied to surface treatment of not only a display surface of a smartphone, a tablet terminal, or the like but also various other materials. There is also a demand for further improvement in durability of surface treatment agents.

The present invention is directed to providing a fluorine-containing ether compound, a fluorine-containing ether composition, and a coating liquid that each can form a surface layer that excels in durability; an article having a surface layer that excels in durability; and a compound useful as a source material of a fluorine-containing ether compound.

The present invention provides a fluorine-containing ether compound and a method of manufacturing the same, a compound and a method of manufacturing the same, a fluorine-containing ether composition, a coating liquid, and an article and a method of manufacturing the same having any of the following constitutions [1] to [10].

[1] A fluorine-containing ether compound expressed by the following formula (A1) or (A2):

{R^{f}O—(R^{f1}O)ₘ₁—(R¹)ₘ₂—(CHF)ₘ₃—O—(CHF)ₘ₄}ₙ₁-Q¹(-T¹)ₙ₂ Formula (A1)

(T²)ₙ₃-Q²-(CHF)ₘ₅—O—(CHF)ₘ₆—(R²)ₘ₇—O—(R^{f2}O)ₘ₈—(R³)ₘ₉—(CHF)ₘ₁₀—O—(CHF)ₘ₁₁-Q³(-T³)ₙ₄ Formula (A2),
wherein
R^{f} is a fluoroalkyl group having a carbon number of from 1 to 20, and when there are a plurality of R^{f}, the plurality of R^{f} may be identical to or different from each other,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom, and when there are a plurality of R¹, the plurality of R¹ may be identical to or different from each other,
Q¹ is an (n1+n2)-valent linking group,
Q² is a (1+n3)-valent linking group,
Q³ is a (1+n4)-valent linking group,
T¹, T², and T³ are each independently an adhesive group, and when there are a plurality of T¹, a plurality of T², or a plurality of T³, the plurality of T¹, the plurality of T², or the plurality of T³ may each be identical to or different from each other,
m1 and m8 are each independently an integer of from 0 to 210, and when there are a plurality of m1, the plurality of m1 may be identical to or different from each other,
m2, m7, and m9 are each independently 0 or 1, and when there are a plurality of m2, the plurality of m2 may be identical to or different from each other,
m3 and m4 are each independently 0 or 1, m3+m4 is 1 or 2, and when there are a plurality of (CHF)ₘ₃—O—(CHF)ₘ₄, the combinations of m3 and m4 may be identical to or different from each other,
m1+m2 is 1 or greater,
m7+m8+m9 is 1 or greater,
m5 and m6 are each independently 0 or 1, and m5+m6 is 1 or 2,
m10 and m11 are each independently 0 or 1, and m10+m11 is 1 or 2,
n1 is an integer of from 1 to 10, and
n2, n3, and n4 are each independently an integer of from 1 to 20.

[2] The fluorine-containing ether compound of [1], wherein the m3, m6, or m10 is 1.

[3] The fluorine-containing ether compound of [1] or [2], wherein the m4, m5, or m11 is 0.

[4] A compound expressed by the following formula (B1) or (B2):

R^{f}O—(R^{f1}O)ₘ₁—(R¹)ₘ₂—CHO Formula (B1)

OHC—(R²)ₘ₇—O—(R^{f2}O)ₘ₈—(R³)ₘ₉—CHO Formula (B2),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
m1 and m8 are each independently an integer of from 0 to 210, and
m2, m7, and m9 are each independently 0 or 1.

[5] A method of manufacturing a fluorine-containing ether compound, the method comprising:
reacting a compound expressed by the following formula (B1) or (B2) and a compound expressed by the following formula (C1):

R^{f}O—(R^{f1}O)ₘ₁—(R¹)ₘ₂—CHO Formula (B1)

OHC—(R²)ₘ₇—O—(R^{f2}O)ₘ₈—(R³)ₘ₉—CHO Formula (B2)

(CH₂═CH—)ₙ₅-Q⁴(—OH)ₙ₆Formula(C1),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
Q⁴ is an (n5+n6)-valent linking group,
m1 and m8 are each independently an integer of from 0 to 210,
m2, m7, and m9 are each independently 0 or 1,
n5 is an integer of from 1 to 20, and
n6 is an integer of from 1 to 10.

[6] A method of manufacturing a compound expressed by the following formula (B1) or (B2), the method comprising:
reacting a compound expressed by the following formula (D1) or (D2) with a silicon hydride compound in the presence of a Lewis acid compound to obtain a compound expressed by the following formula (E1) or (E2);
eliminating Si(R¹⁶)₃ from the compound expressed by the following formula (E1) or (E2) to obtain a compound expressed by the following formula (F1) or (F2); and heating the compound expressed by the following formula (F1) or (F2):

R^{f}O—(R^{f1}O)ₘ₁—(R¹)ₘ₂—C(═O)OR¹¹ Formula (D1)

R¹¹OC(═O)—(R²)ₘ₇—O—(R^{f2}O)ₘ₈—(R³)ₘ₉—C(═O)OR¹¹ Formula (D2)

R^{f}O—(R^{f1}O)ₘ₁—(R¹)ₘ₂—CH(OR¹²)₂ Formula (E1)

(R¹²O)₂CH—(R²)ₘ₇—O—(R^{f2}O)ₘ₈—(R³)ₘ₉—CH(OR¹³)₂ Formula (E2)

R^{f}O—(R^{f1}O)ₘ₁—(R¹)ₘ₂—CH(OR¹⁴)₂ Formula (F1)

(R¹⁴O)₂CH—(R²)ₘ₇—O—(R^{f2}O)ₘ₈—(R³)ₘ₉—CH(OR¹⁵)₂ Formula (F2)

R^{f}O—(R^{f1}O)ₘ₁—(R¹)ₘ₂—CHO Formula (B1)

OHC—(R²)ₘ₇—O—(R^{f2}O)ₘ₈—(R³)ₘ₉—CHO Formula (B2),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
R¹¹ is an alkyl group that may have a hydrogen atom or a substituent, and when there are a plurality of R¹¹, the plurality of R¹¹ may be identical to or different from each other,
R¹² and R¹³ are each independently Si(R¹⁶)₃ or R¹¹, and at least one of the plurality of R¹² and at least one of the plurality of R¹³ are Si(R¹⁶)₃,
R¹⁴ and R¹⁵ are each independently a hydrogen atom or R¹¹, and at least one of the plurality of R¹⁴ and at least one of the plurality of R¹⁵ are a hydrogen atom,
R¹⁶ is a hydrogen atom, a halogen atom, an alkyl group, an aryl group, or an alkoxy group, and the plurality of R¹⁶ may be identical to or different from each other,
m1 and m8 are each independently an integer of from 0 to 210, and
m2, m7, and m9 are each independently 0 or 1.

[7] A fluorine-containing ether composition comprising:
one or more kinds of the fluorine-containing ether compound of any one of [1] to [3]; and
another fluorine-containing ether compound.

[8] A coating liquid comprising:
the fluorine-containing ether compound of any one of [1] to [3] or the fluorine-containing ether composition of [7]; and
a liquid medium.

[9] An article comprising:
a surface layer formed of the fluorine-containing ether compound of any one of [1] to [3] or the fluorine-containing ether composition of [7].

[10] A method of manufacturing an article, the method comprising:
forming a surface layer through a dry coating technique or a wet coating technique with use of the fluorine-containing ether compound of any one of [1] to [3], the fluorine-containing ether composition of [7], or the coating liquid of [8].

The above and other objects, features and advantages of the present disclosure will become more fully understood from the detailed description given hereinbelow and the accompanying drawings which are given by way of illustration only, and thus are not to be considered as limiting the present disclosure.

The present invention provides a fluorine-containing ether compound, a fluorine-containing ether composition, and a coating liquid that each can form a surface layer that excels in durability; an article having a surface layer that excels in durability; and a compound useful as a source material of a fluorine-containing ether compound.

### BRIEF DESCRIPTION OF THE DRAWINGS

FIG. 1 is a schematic sectional view illustrating an example of an article according to the present invention.

### DESCRIPTION OF EMBODIMENTS

In the present specification, a compound expressed by Formula (A1) is referred to as Compound (A1). This convention applies similarly to compounds expressed by other formulas.

In the present specification, the terms listed below are to be construed as stated below.

The term “reactive silyl group” is a collective term for a hydrolytic silyl group and a silanol group (Si—OH). The term “hydrolytic silyl group” means a group that can form a silanol group through a hydrolysis reaction.

The term “surface layer” means a layer formed on a surface of a substrate.

When a fluorine-containing ether compound is a mixture of a plurality of fluorine-containing ether compounds each having a polyfluoropolyether chain of a different chain length, the “molecular weight” of the polyfluoropolyether chains is a number-average molecular weight calculated by obtaining the number (mean value) of oxyfluoroalkylene units through ¹H-NMR and ¹⁹F-NMR.

When a fluorine-containing ether compound is a fluorine-containing ether compound having a polyfluoropolyether chain of a single chain length, the “molecular weight” of the polyfluoropolyether chain is a molecular weight calculated by determining the structure of the polyfluoropolyether chain through ¹H-NMR and ¹⁹F-NMR.

The terms “from” and “to” indicating a numerical value range mean that each includes the numerical value following it as the lower limit value or the upper limit value.

[Fluorine-Containing Ether Compound]A fluorine-containing ether compound according to the present invention (also referred to below as the present compound) is a fluorine-containing ether compound expressed by the following formula (A1) or (A2).

{R^{f}O—(R^{f1}O)ₘ₁—(R¹)ₘ₂—(CHF)ₘ₃—O—(CHF)ₘ₄}ₙ₁-Q¹(-T¹)ₙ₂ Formula (A1)

(T²)ₙ₃-Q²-(CHF)ₘ₅—O—(CHF)ₘ₆—(R²)ₘ₇—O—(R^{f2}O)ₘ₈—(R³)ₘ₉—(CHF)ₘ₁₀—O—(CHF)ₘ₁₁-Q³(-T³)ₙ₄ Formula (A2)

In the above,
R^{f} is a fluoroalkyl group having a carbon number of from 1 to 20, and when there are a plurality of R^{f}, the plurality of R^{f} may be identical to or different from each other,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom, and when there are a plurality of R¹, the plurality of R¹ may be identical to or different from each other,
Q¹ is an (n1+n2)-valent linking group,
Q² is a (1+n3)-valent linking group,
Q³ is a (1+n4)-valent linking group,
T¹, T², and T³ are each independently an adhesive group, and when there are a plurality of T¹, a plurality of T², or a plurality of T³, the plurality of T¹, the plurality of T², or the plurality of T³ may each be identical to or different from each other,
m1 and m8 are each independently an integer of from 0 to 210, and when there are a plurality of m1, the plurality of m1 may be identical to or different from each other,
m2, m7, and m9 are each independently 0 or 1, and when there are a plurality of m2, the plurality of m2 may be identical to or different from each other,
m3 and m4 are each independently 0 or 1, m3+m4 is 1 or 2, and when there are a plurality of (CHF)ₘ₃—O—(CHF)ₘ₄, the combinations of m3 and m4 may be identical to or different from each other,
m1+m2 is 1 or greater,
m7+m8+m9 is 1 or greater,
m5 and m6 are each independently 0 or 1, and m5+m6 is 1 or 2,
m10 and m11 are each independently 0 or 1, and m10+m11 is 1 or 2,
n1 is an integer of from 1 to 10, and
n2, n3, and n4 are each independently an integer of from 1 to 20.

The above-described present compound has a polyfluoropolyether chain [R^{f}O—(R^{f1}O)ₘ₁—(R¹)ₘ₂] or [(R²)ₘ₇—O—(R^{f2}O)ₘ₈—(R³)ₘ₉], an adhesive group [T], and a specific linking part [—(CHF)ₚ₁—O—(CHF)ₚ₂-Q^{p3}] (in which p1 is m3, m6, or m10; p2 is m4, m5, or m11; and p3 is 1, 2, or 3) that links the polyfluoropolyether chain and the adhesive group.

Compound (A1) is a compound having a structure of “n1 monovalent polyfluoropolyether chain-linking group-adhesive group,” and Compound (A2) is a compound having a structure of “adhesive group-linking group-divalent polyfluoropolyether chain-linking group-adhesive group.”

The present compound has a polyfluoropolyether chain. The present compound having a polyfluoropolyether chain provides a surface layer that excels in fingerprint stain removability. Furthermore, the present compound has two or more adhesive groups at at least one of the terminals. The present compound having two or more adhesive groups at a terminal chemically bonds with a substrate strongly and thus provides a surface layer that excels in friction resistance. Moreover, the present compound has at least one —O—CHF-between a polyfluoropolyether chain and a linking group Q^{p3} in the linking group. The present inventors have found that disposing the substituent —O—CHF— adjacent to the linking group Q^{p3} can bring about excellent durability of the present compound on a surface of a substrate and thus have completed the present invention. Although the workings with which this substituent improves durability is not yet fully understood, in one speculation, the hydrogen atom in —O—CHF— may have a more positive electric charge than the hydrogen atoms in —O—CH₂— due to the workings of the oxygen atom and the fluorine atom. In this speculation, this hydrogen atom forms a hydrogen bond with the fluorine atom in —O—CHF— of an adjacent molecule, and thus a hydrogen bond network like FCH . . . FCH . . . FCH is formed. Thus, the adhesiveness of a film may improve, and high durability may be obtained. Herein, “ . . . ” represents a hydrogen bond. When the substituent —O—CHF— is disposed adjacent to the linking group Q^{p3}, flexibility of a polyfluoropolyether chain after a surface layer has been formed is retained, as compared to a case where the substituent —O—CHF— is disposed inside a polyfluoropolyether chain. It is, therefore, speculated that a surface layer with a large water contact angle can be obtained.

In this manner, a surface layer formed of the present compound has characteristics of having a large water contact angle, excelling in removability in fingerprints or the like, and excelling in durability, such as friction resistance, chemical resistance, or light resistance.

Since R^{f} is a fluoroalkyl group having a carbon number of from 1 to 20, a surface layer further excels in friction resistance or fingerprint stain removability. From the standpoint of a surface layer further excelling in friction resistance or fingerprint stain removability, the carbon number of the fluoroalkyl group of R^{f} is preferably from 1 to 6, more preferably from 1 to 4, or particularly preferably from 1 to 3.

From the standpoint of a surface layer further excelling in friction resistance or fingerprint stain removability, the fluoroalkyl group of R^{f} is preferably a perfluoroalkyl group. Compound (A1) in which R^{f} is a perfluoroalkyl group has CF₃— at a terminal. Compound (A1) in which a terminal is CF₃— can form a surface layer of low surface energy, and thus the surface layer further excels in friction resistance or fingerprint stain removability.

Examples of the fluoroalkyl group of R^{f} include CF₃—, CF₃CF₂—, CF₃CF₂CF₂—, CF₃CF₂CF₂CF₂—, CF₃CF₂CF₂CF₂CF₂—, CF₃CF₂CF₂CF₂CF₂CF₂—, or CF₃CF(CF₃)—.

From the standpoint of a surface layer further excelling in friction resistance or fingerprint stain removability, (R^{f1}O)ₘ₁ and (R^{f2}O)ₘ₈ preferably have a structure expressed by the following formula (R^{f}-1).

(R^{f11}O)ₖ₁(R^{f12}O)ₖ₂(R^{f13}O)ₖ₃(R^{f14}O)ₖ₄(R^{f15}O)ₖ₅(R^{f16}O)ₖ₆ Formula (R^{f}-1)

In the above,
R^{f11} is a fluoroalkylene group having a carbon number of 1,
R^{f12} is a fluoroalkylene group having a carbon number of 2,
R^{f13} is a fluoroalkylene group having a carbon number of 3,
R^{f14} is a fluoroalkylene group having a carbon number of 4,
R^{f15} is a fluoroalkylene group having a carbon number of 5,
R^{f16} is a fluoroalkylene group having a carbon number of 6, and
k1, k2, k3, k4, k5, and k6 each independently represent 0 or an integer no smaller than 1; k1+k2+k3+k4+k5+k6 is an integer of from 0 to 210; and when there are a plurality of R^{f11} to R^{f16}, each plurality of R^{f11} to R^{f16} may be identical to or different from each other.

Herein, the order of bonding of (R^{f11}O) to (R^{f16}O) in Formula (R^{f}-1) is flexible. In Formula (R-1), k1 to k6 each represent the number of (R^{f11}O) to (R^{f16}O), respectively, and do not represent their arrangement. For example, (R^{f15}O)ₖ₅ indicates that the number of (R^{f15}O) is k5 and does not represent the block arrangement structure of (R^{f15}O)ₖ₅. In a similar manner, the order in which (R^{f11}O) to (R^{f16}O) are listed does not represent the bonding order of the respective units.

The fluoroalkylene group having a carbon number of from 3 to 6 may be a linear fluoroalkylene group or a fluoroalkylene group having a branch or a ring structure.

Specific examples of R^{f11} include CHF or CF₂. Specific examples of R^{f12} include CF₂CF₂, CF₂CHF, or CF₂CH₂. Specific examples of R^{f13} include CF₂CF₂CF₂, CF₂CF₂CHF, CF₂CHFCF₂, CF₂CF₂CH₂, CF₂CH₂CF₂, or CF(CF₃)CF₂. Specific examples of R^{f14} include CF₂CF₂CF₂CF₂, CF₂CF₂CF₂CH₂, CHFCF₂CF₂CF₂, CF₂CH₂CF₂CF₂, CF(CF₃)CF₂CF₂, or a perfluorocyclobutane-1,2-diyl group. Specific examples of R¹⁵ include CF₂CF₂CF₂CF₂CF₂, CF₂CF₂CF₂CF₂CH₂, CHFCF₂CF₂CF₂CF₂, or CF₂CF₂CH₂CF₂CF₂. Specific examples of R^{f16} include CF₂CF₂CF₂CF₂CF₂CF₂, CF₂CF₂CF₂CF₂CF₂CH₂, or CF₂CF₂CF₂CF₂CF₂CHF.
(R^{f1}O)ₘ₁ and (R^{f2}O)ₘ₈ preferably have at least in part thereof a structure indicated below:
{(OCF₂)ₖ₁₁ (OCF₂CF₂)ₖ₁₂},
(OCF₂CF₂)ₖ₁₃,
(OCF₂CF₂CF₂)ₖ₁₄,
(OCF₂CF₂—OCF₂CF₂CF₂CF₂)ₖ₁₅,
(OCF₂CF₂CF₂)ₖ₁₆(OCF₂CF₂)ₖ₁₇,
(OCF₂CF₂CF₂CF₂CF₂)ₖ₁₆(OCF₂)ₖ₁₇,
(OCF₂CF₂CF₂CF₂CF₂)ₖ₁₆(OCF₂CF₂)ₖ₁₇,
(OCF₂CF₂CF₂CF₂CF₂CF₂)ₖ₁₆(OCF₂)ₖ₁₇,
(OCF₂CF₂CF₂CF₂CF₂CF₂)ₖ₁₆(OCF₂CF₂)ₖ₁₇,
(OCF₂CF₂CF₂CF₂CF₂—OCF₂)ₖ₁₈,
(OCF₂CF₂CF₂CF₂CF₂—OCF₂CF₂)ₖ₁₈,
(OCF₂CF₂CF₂CF₂CF₂CF₂—OCF₂)ₖ₁₈,
(OCF₂CF₂CF₂CF₂CF₂CF₂—OCF₂CF₂)ₖ₁₈,
(OCF₂—OCF₂CF₂CF₂CF₂CF₂)ₖ₁₈,
(OCF₂—OCF₂CF₂CF₂CF₂CF₂CF₂)ₖ₁₈,
(OCF₂CF₂—OCF₂CF₂CF₂CF₂CF₂)ₖ₁₈,
(OCF₂CF₂—OCF₂CF₂CF₂CF₂CF₂CF₂)ₖ₁₈,
(OCF(CF₃)CF₂)ₖ₁₉, or
(OCF₂CF(CF₃))ₖ₂₀(OCF₂CF₂CF₂)ₖ₂₁(OCF₂CF₂)ₖ₂₂.

In the above, k11, k12, k13, k14, k15, k16, k17, k18, k19, k20, k21, and k22 are each an integer no smaller than 1, and the upper limit value is adjusted in accordance with the upper limit value of m1 or m8.

Herein, {(OCF₂)ₖ₁₁(OCF₂CF₂)ₖ₁₂} indicates that k11 (OCF₂) and k12 (OCF₂CF₂) are disposed at random.

Herein, m1 and m8 are each independently from 0 to 210. When m1 is 0, (R^{f1}O)ₘ₁ represents a single bond. Meanwhile, when m8 is 0, (R^{f2}O)ₘ₈ represents a single bond. From the standpoint of water repellency or the like of a surface layer to be obtained, m1 and m8 are preferably from 1 to 210 or more preferably from 2 to 210.

From the standpoint of various characteristics of the fluorine-containing ether compound, such as chemical resistance or a low refractive index property, (R^{f1}O)ₘ₁ and (R^{f2}O)ₘ₈ preferably have a fluorination yield expressed by the following equation (1) of no lower than 60%, more preferably no lower than 80%, or even more preferably substantially 100%, that is, perfluoroether.

fluorination yield(%)=(number of fluorine atoms)/{(number of fluorine atoms)+(number of hydrogen atoms)}×100 Equation (1)

R¹, R², and R³ are each independently an alkylene group that may have a fluorine atom. From the standpoint of a surface layer further excelling in friction resistance or fingerprint stain removability, R¹ to R³ are preferably a fluoroalkylene group. The alkylene group of R¹ to R³ is preferably a linear fluoroalkylene group. Meanwhile, the carbon number of the alkylene group of R¹ to R³ is preferably from 1 to 6.

Herein, m2, m7, and m9 are each independently 0 or 1. When m2 is 0, (R¹)ₘ₂ represents a single bond. When m7 is 0, (R²)ₘ₇ represents a single bond. Meanwhile, when m9 is 0, (R³)ₘ₉ represents a single bond. From the standpoint of ease of synthesizing the present compound or the like, m2, m7, and m9 are preferably 1.

The present compound has a linking part [—(CHF)ₚ₁—O—(CHF)ₚ₂-Q^{p3}] (in which p1 is m3, m6, or m10; p2 is m4, m5, or m11; and p3 is 1, 2, or 3) that links a polyfluoropolyether chain and an adhesive group T. As p1+p2 is 1 or 2, the hydrogen bond network described above can be formed. From the standpoint of water repellency and durability of a surface layer to be obtained, p1 (m3, m6, or m10) is preferably 1. Meanwhile, from the standpoint of ease of synthesizing the present compound or the like, p2 (m4, m5, or m11) is preferably 0.

Q¹ is an (n1+n2)-valent linking group and links n2 adhesive groups to n1 polyfluoropolyether chains. Q² is a (1+n3)-valent linking group, and Q³ is a (1+n4)-valent linking group and links n3 adhesive groups to one terminal of a polyfluoropolyether chain and links n4 adhesive groups to the other terminal of the polyfluoropolyether chain. When Q¹, Q², and Q³ (these may also be referred to collectively as Q^{p3}) are divalent linking groups, these Q^{p3} may be a single bond or a linear linking group. Examples of a linear linking group include an alkylene group that may have a substituent. The alkylene group of Q^{p3} is preferably an alkylene group having a carbon number of from 1 to 6. Examples of a substituent that the alkylene group may have include a fluorine atom or an alkyl group.

When Q^{p3} is a trivalent or higher linking group, this linking group is a group having a branch point P. Examples of a branch point P include C, N, Si, a ring structure, or an organopolysiloxane residue.

From the standpoint of ease of manufacturing the present compound or from the standpoint of durability or the like of a surface layer formed of the present compound, a ring structure constituting the branch point is preferably one selected from the group consisting of an aliphatic ring of from 3 to 8 members, an aromatic ring of from 3 to 8 members, a hetero ring of from 3 to 8 members, and a fused ring composed of two or more of the rings listed above, and ring structures indicated by the following formulas are particularly preferable. Herein, the ring structure may have a substituent, such as a halogen atom, an alkyl group (may include an ether oxygen atom between a carbon atom and another carbon atom), a cycloalkyl group, an alkenyl group, an allyl group, an alkoxy group, or an oxo group (═O).

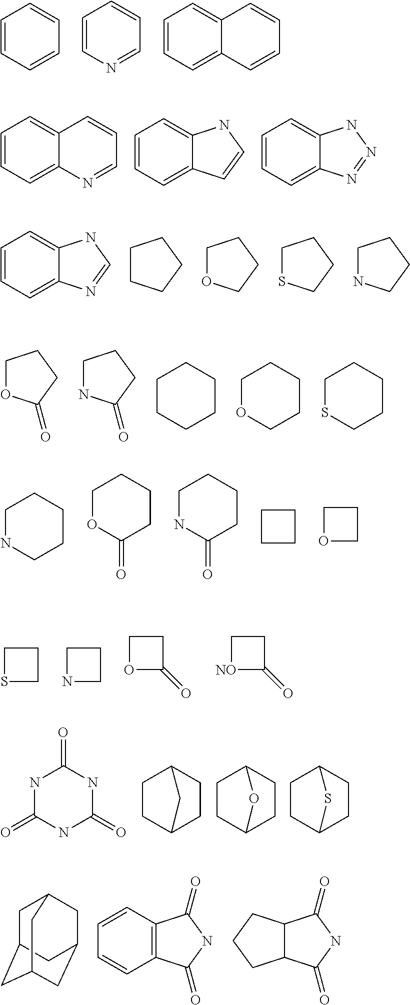

Examples of an organopolysiloxane residue constituting the branch point include the following groups. Herein, R⁵ in the following formulas is a hydrogen atom, an alkyl group, an alkoxy group, or a phenyl group. The carbon number of the alkyl group or the alkoxy group of R⁵ is preferably from 1 to 10 or particularly preferably 1.

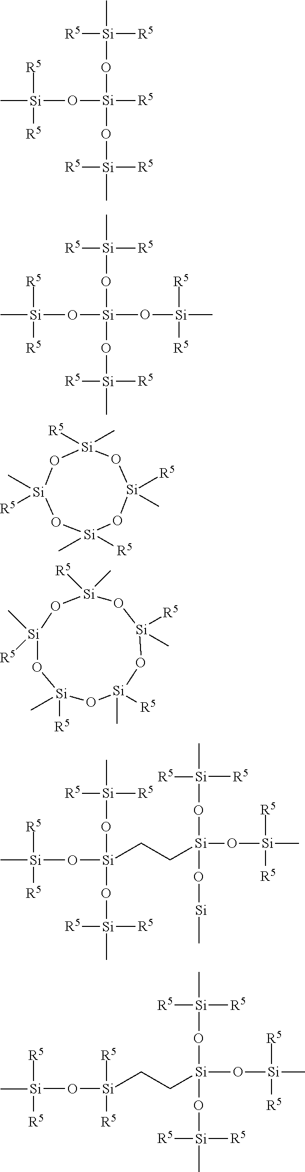

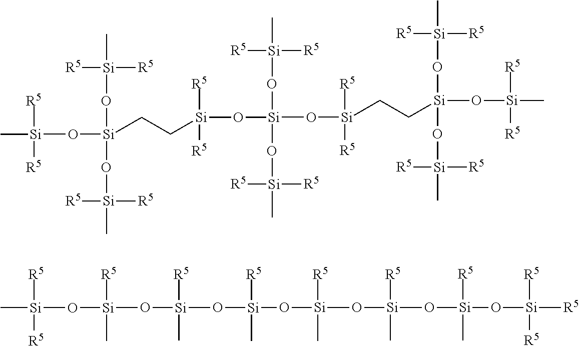

Q^{p3} may have at least one bond B selected from the group consisting of —C(O)NR—, —C(O)O—, —C(O)—, —O—, —NR⁶—, —S—, —OC(O)O—, —NHC(O)O—, —NHC(O)NR⁶—, —SO₂NR⁶—, —Si(R⁶)₂—, —OSi(R⁶)₂—, —Si(CH₃)₂-Ph-Si(CH₃)₂—, and a divalent organopolysiloxane residue. From the standpoint ease of manufacturing the present compound, Q^{p3} may preferably have at least one bond selected from the group consisting of —C(O)NR—, —C(O)—, —NR⁶—, and —O—, and from the standpoint of a surface layer excelling in light resistance and chemical resistance, —C(O)NR— or —C(O)— is more preferable. R⁶ in the above bond is a hydrogen atom or an alkyl group or a phenyl group having a carbon number of from 1 to 6, and Ph is a phenylene group. From the standpoint of ease of manufacturing the present compound, the carbon number of the alkyl group of R⁶ is preferably from 1 to 3 or particularly preferably from 1 to 2.

Examples of a divalent organopolysiloxane residue include the groups of the following formulas. Herein, R⁷ in the following formulas is a hydrogen atom, an alkyl group, an alkoxy group, or a phenyl group.

The carbon number of the alkyl group or the alkoxy group of R⁷ is preferably from 1 to 10 or particularly preferably 1.

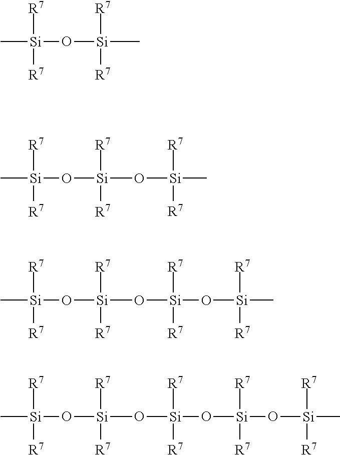

Examples of Q^{p3} include a combination of two or more divalent hydrocarbon groups and one or more branch points P or a combination of two or more hydrocarbon groups, one or more branch points P, and one or more bonds B.

Examples of a divalent hydrocarbon group include a divalent aliphatic hydrocarbon group (alkylene group, cycloalkylene group, etc.) or a divalent aromatic hydrocarbon group (phenylene group, etc.). The carbon number of a divalent hydrocarbon group is preferably from 1 to 10, more preferably from 1 to 6, or particularly preferably from 1 to 4.

From the standpoint of ease of manufacturing the present compound, the Q^{p3} is preferably a group expressed by any one of the following formulas (Q1) to (Q7).

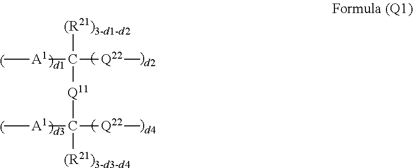

(-A¹-)ₑ₁C(R²²)₄₋ₑ₁₋ₑ₂(-Q²²-)ₑ₂ Formula (Q²)

-A²-N(-Q²³-)₂ Formula (Q³)

(-A³-)ₕ₁Z¹(-Q²⁴-)ₕ₂ Formula (Q⁴)

(-A²-)ᵢ₁Si(R²³)₄₋ᵢ₁₋ᵢ₂(-Q²⁵-)ᵢ₂Formula(Q⁵)

-A¹-Q²⁶- Formula (Q⁶)

-A¹-CH(-Q²²-)—Si(R²³)₃₋ᵢ₃(-Q²⁵-)ᵢ₃ Formula (Q⁷)

In Formulas (Q1) to (Q7), the side where A¹, A², or A³ is present connects to a polyfluoropolyether chain, and the side where Q²², Q²³, Q²⁴, Q²⁵, or Q²⁶ is present connects to an adhesive group T¹, T², or T³,
A¹ is a single bond, an alkylene group, or a group having —C(O)NR²⁶—, —C(O)—, —NR²⁶—, or —O— between a carbon atom and another carbon atom of an alkylene group having a carbon number of two or greater,
A² is an alkylene group or a group having —C(O)NR²⁶—, —C(O)—, —NR²⁶—, or —O— between a carbon atom and another carbon atom of an alkylene group having a carbon number of two or greater,
A³ is A¹ when an atom in Z¹ with which A³ bonds is a carbon atom or is A² when an atom in Z¹ with which A³ bonds is a nitrogen atom,
Q¹¹ is a single bond, —O—, an alkylene group, or a group having —C(O)NR²⁶—, —C(O)—, —NR²⁶—, or —O— between a carbon atom and another carbon atom of an alkylene group having a carbon number of two or greater,
Q²² is an alkylene group; a group having —C(O)NR²⁶—, —C(O)—, —NR²⁶—, or —O— between a carbon atom and another carbon atom of an alkylene group having a carbon number of two or greater; a group having —C(O)NR²⁶—, —C(O)—, —NR²⁶—, or —O— at the terminal of an alkylene group where the alkylene group does not connect to an adhesive group T; or a group having —C(O)NR²⁶—, —C(O)—, —NR²⁶—, or —O—between a carbon atom and another carbon atom of an alkylene group having a carbon number of two or greater and having —C(O)NR²⁶—, —C(O)—, —NR²⁶—, or —O— at the terminal where the alkylene group does not connect to an adhesive group T, and when Q^{p3} has two or more Q²², the two or more Q²² may be identical to or different from each other,
Q²³ is an alkylene group or a group having —C(O)NR²⁶—, —C(O)—, —NR²⁶—, or —O— between a carbon atom and another carbon atom of an alkylene group having a carbon number of two or greater, and the two or more Q²³ may be identical to or different from each other,
Q²⁴ is Q²² when an atom in Z¹ with which Q²⁴ bonds is a carbon atom or is Q²³ when an atom in Z¹ with which Q²⁴ bonds is a nitrogen atom, and when Q^{p3} has two or more Q²⁴, the two or more Q²⁴ may be identical to or different from each other,
Q²⁵ is an alkylene group or a group having —C(O)NR²⁶—, —C(O)—, —NR²⁶—, or —O— between a carbon atom and another carbon atom of an alkylene group having a carbon number of two or greater, and when Q^{p3} has two or more Q²⁵, the two or more Q²⁵ may be identical to or different from each other,
Q²⁶ is an alkylene group or a group having —C(O)NR²⁶—, —C(O)—, —NR²⁶—, or —O— between a carbon atom and another carbon atom of an alkylene group having a carbon number of two or greater,
Z¹ is a group that has a carbon atom or a nitrogen atom with which A³ directly bonds and has an (h1+h2)-valent ring structure having a carbon atom or a nitrogen atom with which Q²⁴ directly bonds,
R²¹ is a hydrogen atom or an alkyl group, and when Q^{p3} has two or more R²¹, the two or more R²¹ may be identical to or different from each other,
R²² is a hydrogen atom, a hydroxyl group, an alkyl group, or an acyloxy group, and when Q^{p3} has two or more R²², the two or more R²² may be identical to or different from each other,
R²³ is an alkyl group, and when Q^{p3} has two or more R²³, the two or more R²³ may be identical to or different from each other,
R²⁶ is a hydrogen atom or an alkyl group or a phenyl group having a carbon number of from 1 to 6,
d1 is an integer of from 0 to 3, d2 is an integer of from 0 to 3, and d1+d2 is an integer of from 1 to 3,
d3 is an integer of from 0 to 3, d4 is an integer of from 0 to 3, and d3+d4 is an integer of from 1 to 3,
d1+d3 is an integer of from 1 to 5 in Q¹ and is 1 in Q² and Q³,
d2+d4 is an integer of from 1 to 5,
e1 an integer of from 1 to 3 in Q¹ and is 1 in Q² and Q³,
e2 is an integer of from 1 to 3,
e1+e2 is an integer of from 2 to 4,
h1 is an integer no smaller than 1 in Q¹ and is 1 in Q² and Q³,
h2 is an integer no smaller than 1,
i1 is an integer of from 1 to 3 in Q¹ and is 1 in Q² and Q³,
i2 is an integer of from 1 to 3,
i1+i2 is an integer of from 2 to 4, and
i3 is an integer of from 1 to 3.

From the standpoint of ease of manufacturing the present compound or from the standpoint of a surface layer further excelling in friction resistance, light resistance, or chemical resistance, the carbon number of the alkylene group of Q¹¹, Q²², Q²³, Q²⁴, Q²⁵, or Q²⁶ is preferably from 1 to 10, more preferably from 1 to 6, or particularly preferably from 1 to 4. However, when the alkylene group has a specific bond between a carbon atom and another carbon atom, the lower limit value of the carbon number of the alkylene group is 2.

Examples of a ring structure in Z¹ include the ring structures described above, and preferable modes are also similar.

From the standpoint of ease of manufacturing the present compound, the carbon number of the alkyl group of R²¹, R²², or R²³ is preferably from 1 to 6, more preferably from 1 to 3, or particularly preferably from 1 to 2.

From the standpoint of ease of manufacturing the present compound, the carbon number of the alkyl group portion of the acyloxy group of R²² is preferably from 1 to 6, more preferably from 1 to 3, or particularly preferably from 1 to 2.

From the standpoint of ease of manufacturing the present compound or from the standpoint of a surface layer further excelling in friction resistance or fingerprint stain removability, h1+h2 is preferably from 2 to 6, more preferably from 2 to 4, or particularly preferably 2 to 3.

Other modes of the Q^{p3} include the groups expressed by the following formulas (Q11) to (Q17).

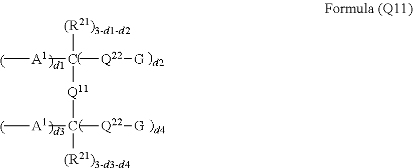

(-A¹-)ₑ₁C(R²²)₄₋ₑ₁₋ₑ₂(-Q²²-G)ₑ₂ Formula (Q12)

-A²-N(-Q²³-G)₂ Formula (Q13)

(-A³-)ₕ₁Z¹(-Q²⁴-G)ₕ₂ Formula (Q14)

(-A²-)ᵢ₁Si(R²³)₄₋ᵢ₁₋ᵢ₂(-Q²⁵-G)ᵢ₂Formula(Q15)

-A¹-Q²⁶-G Formula (Q16)

-A¹-CH(-Q²²-)—Si(R²³)₃₋ᵢ₃(-Q²⁵-G)ᵢ₃ Formula (Q¹⁷)

In Formulas (Q11) to (Q17), the side where A¹, A², or A³ is present connects to a polyfluoropolyether chain, and the side where G is present connects to an adhesive group T¹, T², or T³. G is a group expressed by the following formula (G1), and two or more G of Q^{p3} may be identical to or different from each other. The symbols other than G are the same as the symbols in Formulas (Q¹) to (Q⁷).

—Si(R²⁷)₃₋ⱼ₁(-Q²⁷-)ⱼ₁ Formula (G1)

In Formula (G1), the side where Si is present connects to Q²², Q²³, Q²⁴, Q²⁵, or Q²⁶, and the side where Q²⁷ is present connects to an adhesive group T. R²⁷ is an alkyl group. Q²⁷ is an alkylene group; a group having —C(O)NR²⁸—, —C(O)—, —NR²⁸—, or —O— between a carbon atom and another carbon atom of an alkylene group having a carbon number of two or greater; or —(OSi(R²⁹)₂)ⱼ₂—O—, and the two or more Q²⁷ may be identical to or different from each other. Meanwhile, j1 is 2 to 3. R²⁸ is a hydrogen atom or an alkyl group or a phenyl group having a carbon number of from 1 to 6.

R²⁹ is an alkyl group, a phenyl group, or an alkoxy group, and the two or more R²⁹ may be identical to or different from each other. Meanwhile, j2 is an integer of from 0 to 5, and when j2 is 2 or greater, the two or more (OSi(R²⁹)₂) may be identical to or different from each other.

From the standpoint of ease of manufacturing the present compound or from the standpoint of a surface layer further excelling in friction resistance, light resistance, or chemical resistance, the carbon number of the alkylene group of Q²⁷ is preferably from 1 to 10, more preferably from 1 to 6, or particularly preferably from 1 to 4. However, when the alkylene group has a specific bond between a carbon atom and another carbon atom, the lower limit value of the carbon number of the alkylene group is 2.

From the standpoint of ease of manufacturing the present compound, the carbon number of the alkyl group of R²⁸ is preferably from 1 to 6, more preferably from 1 to 3, or particularly preferably from 1 to 2.

From the standpoint of ease of manufacturing the present compound, the carbon number of the alkyl group of R²⁹ is preferably from 1 to 6, more preferably from 1 to 3, or particularly preferably from 1 to 2.

Meanwhile, j2 is preferably 0 or 1.

T¹, T², and T³ (these may be referred to collectively as T) are groups that are located on the side facing a substrate when a surface layer is formed and exhibits adhesiveness with the substrate. T may form a chemical bond with, for example, a surface of a substrate or may be chemically or physically adsorbed on a surface of a substrate.

From the standpoint of durability, T preferably chemically bonds with a surface of a substrate.

Examples of an adhesive group T include a bromine atom, an iodine atom, a hydroxyl group, an amino group, a carboxy group, an aldehyde group, an epoxy group, a thiol group, a phosphoric acid group, a phosphonic acid group, an unsaturated hydrocarbon group, an aryl group, or a reactive silyl group, and an adhesive group T is preferably selected as appropriate in accordance with the material of a substrate to be applied.

Examples of an amino group for an adhesive group T include —NR³²R³³ (R³² and R³³ are each independently a hydrogen atom or an alkyl group), and from the standpoint of durability of a surface layer, —NH₂ in particular is preferable.

Examples of a phosphoric acid for an adhesive group T include —OP(═O)(OR³⁴)₂ (R³⁴ is a hydrogen atom or an alkyl group, and the plurality of R³⁴ may be identical to or different from each other), and from the standpoint of durability of a surface layer, —OP(═O)(OH)₂ in particular is preferable.

Examples of a phosphonic acid for an adhesive group T include —P(═O)(OR³⁵)₂ (R³⁵ is similar to R³⁴ described above), and from the standpoint of durability of a surface layer, —P(═O)(OH)₂ in particular is preferable.

Examples of an unsaturated hydrocarbon group for an adhesive group T include an acryloyl group, a methacryloyl group, or a vinyl group.

Examples of an aryl group for an adhesive group T include a phenyl group, a naphthyl group, or an anthracenyl group.

Examples of a reactive silyl group for an adhesive group T include a group expressed by —Si(R³¹)_{3-c}(L³¹)_{c}.

L³¹ is a hydrolytic group or a hydroxyl group, and a reactive silyl group is a group in which at least one of a hydrolytic group or a hydroxyl group bonds with a silicon atom.

A hydrolytic group is a group that turns into a hydroxyl group through a hydrolysis reaction. In other words, a hydrolytic silyl group turns into a silanol group (Si—OH) through a hydrolysis reaction. A silanol group further undergoes a dehydration condensation reaction between molecules to form a Si—O—Si bond. Moreover, a silanol group undergoes a dehydration condensation reaction with a hydroxyl group (substrate-OH) on a surface of a substrate to form a chemical bond (substrate-O—Si).

Examples of a hydrolytic group include an alkoxy group, an aryloxy group, a halogen atom, an acyl group, and acyloxy group, or an isocyanate group. The alkoxy group is preferably an alkoxy group having a carbon number of from 1 to 6. The aryloxy group is preferably an aryloxy group having a carbon number of from 3 to 10. The halogen atom is preferably a chlorine atom. The acyl group is preferably an acyl group having a carbon number of from 1 to 6. The acyloxy group is preferably an acyloxy group having a carbon number of from 1 to 6. L³¹ is preferably in particular an alkoxy group having a carbon number of from 1 to 4 or a halogen atom.

From the standpoint of ease of manufacturing the present compound, the hydrolytic group is preferably an alkoxy group or a halogen atom. From the standpoint of lesser outgassing during coating or from the standpoint of excellence in storage stability of the present compound, the hydrolytic group is preferably an alkoxy group having a carbon number of from 1 to 4. The hydrolytic group is particularly preferably an ethoxy group when long-lasting storage stability of the present compound is needed or is particularly preferably a methoxy group when the reaction time after coating is to be shortened.

From the standpoint of ease of manufacturing the present compound, the carbon number of the alkyl group of R³¹ is preferably from 1 to 6, more preferably from 1 to 3, or particularly preferably from 1 to 2.

From the standpoint of greater adhesiveness between a surface layer and a substrate, c is preferably 2 or 3 or more preferably 3.

Some preferred combinations of an adhesive group T and a material of a substrate are illustrated below in the format “adhesive group T-substrate”:
bromine atom-silicon nitride, hydrogen-terminated silicon nitride,
iodine atom-hydrogen-terminated diamond,
hydroxyl group-hydrogen-terminated silicon, halogenated silicon, silicon oxide (SiO₂), aluminum oxide (Al₂O₃), diamond-like carbon (DLC),
amino group-indium tin oxide (ITO), mica,
carboxy group-Al₂O₃, silver oxide (AgO), copper oxide (CuO), zirconium-modified aluminum oxide (Zr/Al₂O₃), amine-terminated oxide (NH₂-terminated oxide), tin oxide (SnO₂),
aldehyde group, epoxy group-hydrogen-terminated silicon, halogenated silicon,
thiol group-gold (Au),
phosphoric acid group, phosphonic acid group-zirconium oxide (ZrO₂), titanium oxide (TiO₂), Al₂O₃, tantalum oxide (Ta₂O₅), Zr/Al₂O₃,
unsaturated hydrocarbon group-hydrogen-terminated diamond, hydrogen-terminated silicon, halogenated silicon, silicon nitride, hydrogen-terminated silicon nitride, polyimide (PI), acryl,
aryl group-Al₂O₃, DLC, or
reactive silyl group-glass, Au, mica, SiO₂, tin oxide (SnO₂), germanium oxide (GeO₂), ZrO₂, TiO₂, Al₂O₃, ITO, stainless steel (SUS), lead zirconate titanate (PZT).

These combinations are not limiting, and the present compound can be used in combination with various other substrates.

Specific examples of the present compound include compounds of the following formulas. The compounds of the following formulas are easy to manufacture industrially, are easy to handle, and provide a surface layer that further excels in water repellency or oil repellency, friction resistance, fingerprint stain removability, lubricity, chemical resistance, light resistance, or chemical resistance. In particular, these compounds especially excel in chemical resistance and are thus preferable.

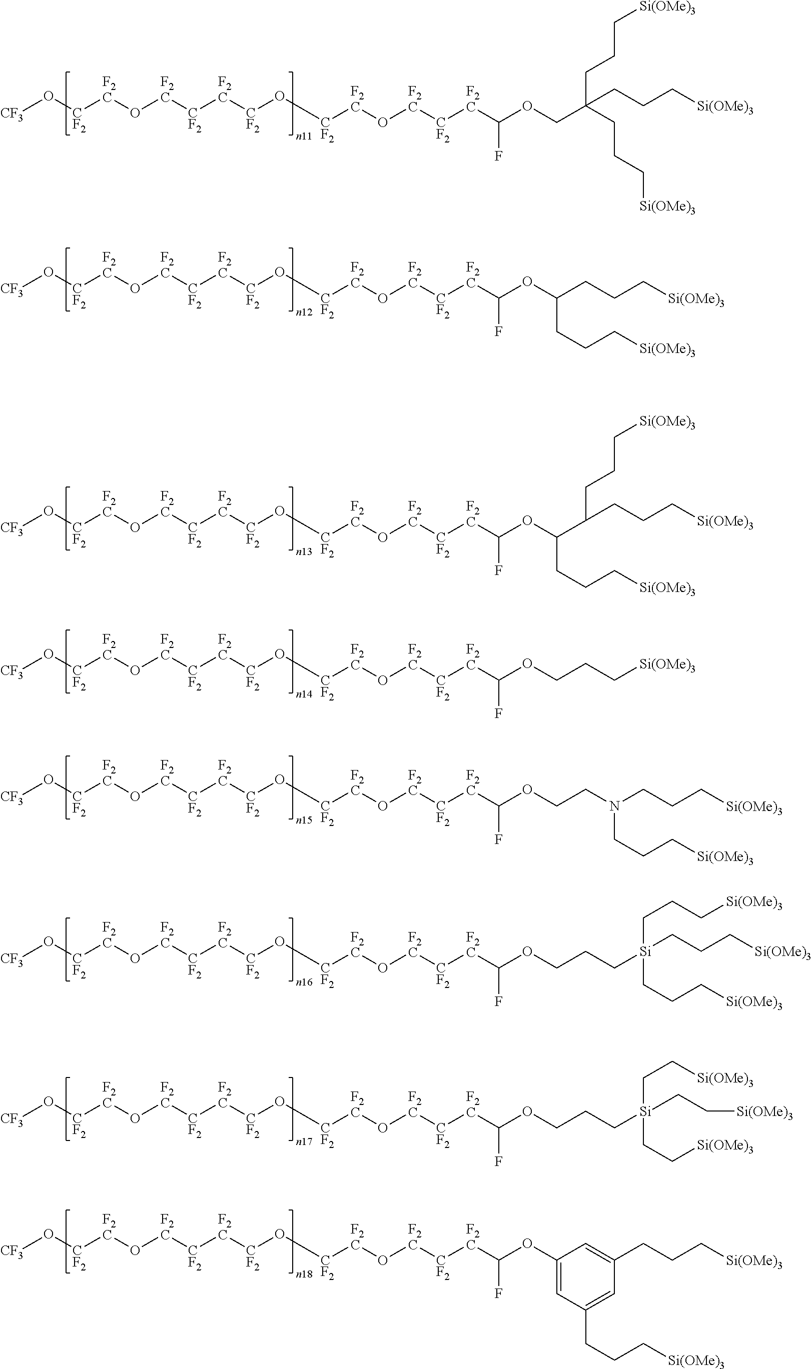

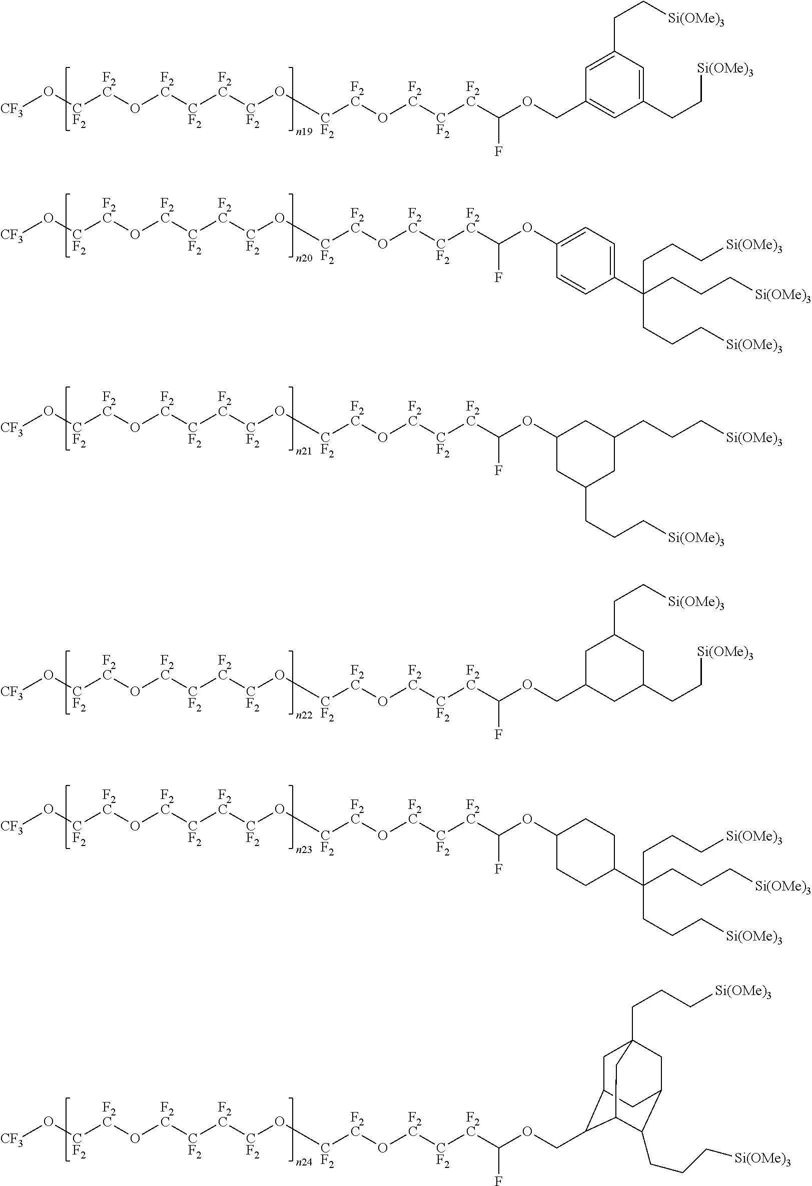

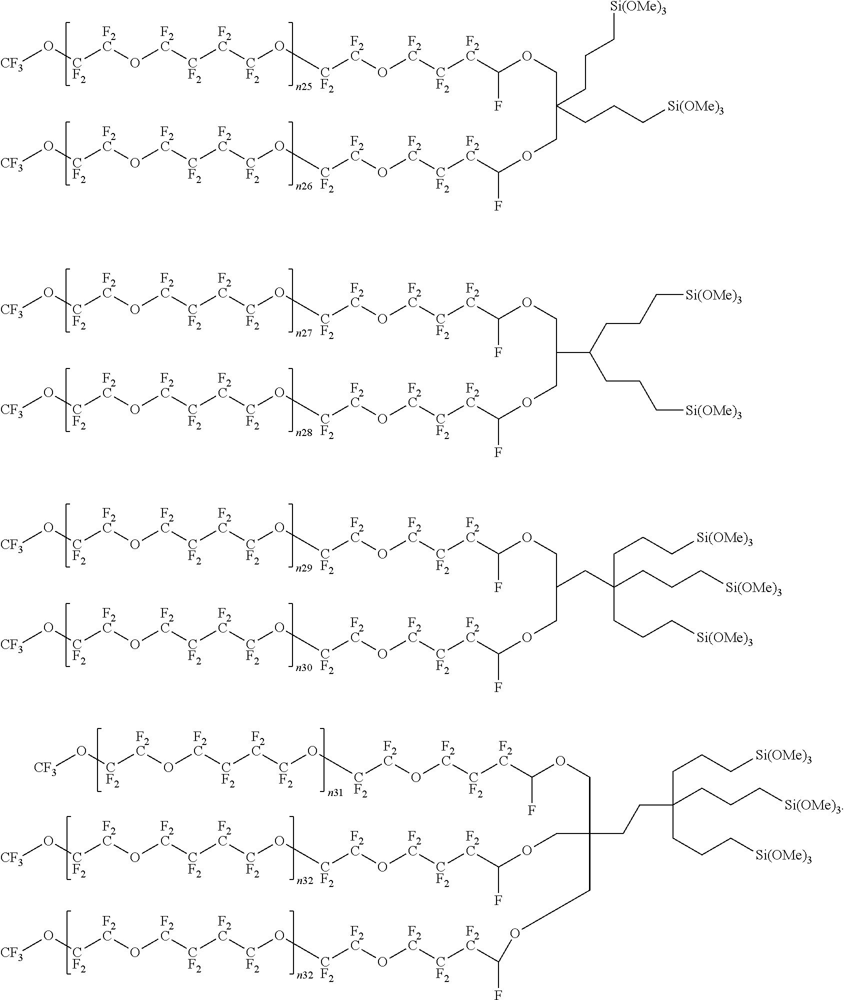

Herein, n11 to n33 are an integer of from 0 to 210.

### (Method of Manufacturing the Present Compound)

Although the method of manufacturing the present compound is not limited to the method described below, the manufacturing method described below can provide the present compound with a high yield. Specifically, a method of manufacturing a fluorine-containing ether compound according to the present invention (also referred to below as the present manufacturing method) is a method of manufacturing a fluorine-containing ether compound expressed by Formula (A1) or Formula (A2), and the method includes reacting a compound expressed by the following formula (B1) or (B2) and a compound expressed by the following formula (C1).

R^{f}O—(R^{f1}O)ₘ₁—(R¹)ₘ₂—CHO Formula (B1)

OHC—(R²)ₘ₇—O—(R^{f2}O)ₘ₈—(R³)ₘ₉—CHO Formula (B2),

(CH₂═CH—)ₙ₅-Q⁴(—OH)ₙ₆ Formula (C1)

In the above, R^{f}, R^{f1}, R^{f2}, R¹, R², R³, m1, m2, m7, m8, and m9 are similar to those in Compound (A1) and Compound (A2) described above, and preferable modes are also similar.
Q⁴ is an (n5+n6)-valent linking group,
n5 is an integer of from 1 to 20, and
n6 is an integer of from 1 to 10.
Q⁴ is a portion that turns into Q¹ of Compound (A1) or into Q² or Q³ of Compound (A2), and preferable modes are similar to those of Q^{p3} described above.

Meanwhile, n5 is a portion that turns into n2 of Compound (A1) or into n3 or n4 of Compound (A2), and preferable modes are also similar.

Meanwhile, n6 is a portion that turns into n1 of Compound (A1), and preferable modes are also similar.

In a case where Compound (B2) and Compound (C1) are to be reacted, a compound in which n6 is 1 is selected.

CH₂═CH— of Compound (C1) corresponds to an adhesive group T of Compound (A1) or Compound (A2). Compounds having another adhesive group may be reacted as necessary to introduce another adhesive group.

For example, when Compound (B1) is reacted with Compound (C1) in which n6 is 1, R^{f}O—(R^{f1}O)ₘ₁—(R¹)ₘ₂—CH(OH)—O-Q⁴-(CH═CH₂)ₙ₅ is obtained. When this compound is fluorinated with a fluorinating agent, R^{f}O—(R^{f1}O)ₘ₁—(R¹)ₘ₂—CHF—O-Q⁴-(CH═CH₂)ₙ₅ is obtained.

Examples of a fluorinating agent include bis(2-methoxyethyl)aminosulfur=trifluoride, diethylaminosulfur trifluoride, perfluoro-1-butanesulfonyl fluoride, 1,1,2,2-tetrafluoro-N,N-dimethylethylamine, (dimethylamino)difluorosulfonium tetrafluoroborate, 4-tert-butyl-2,6-dimethylphenylsulfur trifluoride, 2-chloro-1,3-bis(2,6-diisopropylphenyl)-1H-imidazolium chloride-cesium fluoride, or pyridine-2-sulfonyl fluoride.

In a case where a reactive silyl group, for example, is introduced into —(CH═CH₂)ₙ₅, it can be manufactured through a method in which the aforementioned compound and the following compound 3a are allowed to undergo a hydrosilylation reaction.

HSi(R³¹)_{3-c}(L³¹)_{c} Formula 3a

The symbols in Formula 3a are similar to those of the present compound described above, and preferable modes are also similar. Compound 3a may be synthesized, or a commercially available product may be used.

Compound (B1) and Compound (B2) described above are novel compounds suitable for the present manufacturing method. Although the following method of manufacturing Compound (B1) and Compound (B2) is not limited to the method described below, the manufacturing method described below can provide Compound (B1) and Compound (B2) with a high yield.

Specifically, a method of manufacturing Compound (B1) or Compound (B2) according to the present invention is a method of manufacturing a compound expressed by the following formula (B1) or (B2), and the method includes:
reacting a compound expressed by the following formula (D1) or (D2) with a silicon hydride compound HSi(R¹⁶)₃ in the presence of a Lewis acid compound to obtain a compound expressed by the following formula (E1) or (E2);
eliminating Si(R¹⁶)₃ from the compound expressed by the following formula (E1) or (E2) to obtain a compound expressed by the following formula (F1) or (F2); and
heating the compound expressed by the following formula (F1) or (F2).

R^{f}O—(R^{f1}O)ₘ₁—(R¹)ₘ₂—C(═O)OR¹¹ Formula (D1)

R¹¹OC(═O)—(R²)ₘ₇—O—(R^{f2}O)ₘ₈—(R³)ₘ₉—C(═O)OR¹¹ Formula (D2)

R^{f}O—(R^{f1}O)ₘ₁—(R¹)ₘ₂—CH(OR¹²)₂ Formula (E1)

(R¹²O)₂CH—(R²)ₘ₇—O—(R^{f2}O)ₘ₈—(R³)ₘ₉—CH(OR¹³)₂ Formula (E2)

R^{f}O—(R^{f1}O)ₘ₁—(R¹)ₘ₂—CH(OR¹⁴)₂ Formula (F1)

(R¹⁴O)₂CH—(R²)ₘ₇—O—(R^{f2}O)ₘ₈—(R³)ₘ₉—CH(OR¹⁵)₂ Formula (F2)

R^{f}O—(R^{f1}O)ₘ₁—(R¹)ₘ₂—CHO Formula (B1)

OHC—(R²)ₘ₇—O—(R^{f2}O)ₘ₈—(R³)ₘ₉—CHO Formula (B2)

In the above,
R^{f}, R^{f1}, R^{f2}, R¹, R², R³, m1, m2, m7, m8, and m9 are similar to those of the present compound described above, and preferable modes are also similar,
R¹¹ is an alkyl group that may have a hydrogen atom or a substituent, and when there are a plurality of R¹¹, the plurality of R¹¹ may be identical to or different from each other,
R¹² and R¹³ are each independently Si(R¹⁶)₃ or R¹¹, and at least one of the plurality of R¹² and at least one of the plurality of R¹³ are Si(R¹⁶)₃,
R¹⁴ and R¹⁵ are each independently a hydrogen atom or R¹¹, and at least one of the plurality of R¹⁴ and at least one of the plurality of R¹⁵ are a hydrogen atom, and
R¹⁶ is a hydrogen atom, a halogen atom, an alkyl group, an aryl group, or an alkoxy group, and the plurality of R¹⁶ may be identical to or different from each other.
R¹¹ to R¹⁶ are sites that do not remain in Compound (B1), Compound (B2), or the present compound and may thus be selected as appropriate in accordance with the reactivity or the availability.

The alkyl group of R¹¹ is preferably a methyl group or an ethyl group, and a methyl group is more preferable from the standpoint of reactivity.

R¹⁶ is preferably an alkyl group or more preferably an ethyl group.

Another method of manufacturing the present compound includes a step of reacting a compound expressed by the following formula (B3) or (B4) and a compound expressed by the following formula (C2).

R^{f}O—(R^{f1}O)ₘ₁—(R¹)ₘ₂—OH Formula (B3)

OH—(R²)ₘ₇—O—(R^{f2}O)ₘ₈—(R³)ₘ₉—CHO Formula (B4)

(CH₂═CH—)ₙ₅-Q⁴(—CHO)ₙ₆ Formula (C2)

The symbols in the above formulas are similar to those in Compound (B1), Compound (B2), or Compound (C1) described above, and preferable modes are also similar.

### [Fluorine-Containing Ether Composition]

A fluorine-containing ether composition according to the present invention (also referred to below as the present composition) is a composition that includes the fluorine-containing compound, or the present compound, and at least one of a fluorine-containing compound other than the present compound or an impurity described below. Examples of an impurity include a compound and so on that are inevitable in manufacturing the present compound or another fluorine-containing compound. Herein, the present composition does not include a liquid medium described later.

Examples of another fluorine-containing compound include a fluorine-containing compound produced as a by-product in the process of manufacturing the present compound (also referred to below as a by-product fluorine-containing compound) or a known fluorine-containing compound used for a purpose similar to that of the present compound.

For another fluorine-containing compound, a compound that is less likely to degrade the properties of the present compound is preferable.

From the standpoint of allowing the properties of the present compound to be exhibited sufficiently, the content of another fluorine-containing compound with respect to the total amount of the present composition is preferably lower than 50 mass %, more preferably lower than 30 mass %, or even more preferably lower than 10 mass %.

Examples of a by-product fluorine-containing compound include a fluorine-containing compound that is unreacted in synthesizing the present compound. In a case where the present composition includes a by-product fluorine-containing compound, a purifying process of removing this by-product fluorine-containing compound or reducing the amount of the by-product fluorine-containing compound can be simplified.

Examples of a known fluorine-containing compound include those described in the following literatures:

Perfluoropolyether-modified aminosilane described in Japanese Unexamined Patent Application Publication No. H11-029585,

Silicon-containing organic fluoropolymer described in Japanese Patent No. 2874715,

Organic silicon compound described in Japanese Unexamined Patent Application Publication No. 2000-144097,

Perfluoropolyether-modified aminosilane described in Japanese Unexamined Patent Application Publication No. 2000-327772,

Fluorinated siloxane described in Published Japanese Translation of PCT International Publication for Patent Application, No. 2002-506887,

Organic silicone compound described in Published Japanese Translation of PCT International Publication for Patent Application, No. 2008-534696,

Fluoromodified hydrogenated polymer described in Japanese Patent No. 4138936,

Compound described in U.S. Patent Application Publication No. 2010/0129672, International Patent Publication No. WO2014/126064, or Japanese Unexamined Patent Application Publication No. 2014-070163,

Organosilicon compound described in International Patent Publication No. WO2011/060047 or International Patent Publication No. WO2011/059430,

Fluorine-containing organosilane compound described in International Patent Publication No. WO2012/064649,

Fluorooxyalkylene group-containing polymer described in Japanese Unexamined Patent Application Publication No. 2012-72272,

Fluorine-containing ether compound described in International Patent Publication No. WO2013/042732, International Patent Publication No. WO2013/121984, International Patent Publication No. WO2013/121985, International Patent Publication No. WO2013/121986, International Patent Publication No. WO2014/163004, Japanese Unexamined Patent Application Publication No. 2014-080473, International Patent Publication No. WO2015/087902, International Patent Publication No. WO2017/038830, International Patent Publication No. WO2017/038832, or International Patent Publication No. WO2017/187775,

Perfluoro(poly)ether-containing silane compound described in Japanese Unexamined Patent Application Publication No. 2014-218639, International Patent Publication No. WO2017/022437, International Patent Publication No. WO2018/079743, or International Patent Publication No. WO2018/143433,

Fluoropolyether group-containing polymer-modified silane described in Japanese Unexamined Patent Application Publication No. 2015-199906, Japanese Unexamined Patent Application Publication No. 2016-204656, Japanese Unexamined Patent Application Publication No. 2016-210854, or Japanese Unexamined Patent Application Publication No. 2016-222859, or

Fluorine-containing ether compound described in International Patent Publication No. WO2018/216630, International Patent Publication No. WO2019/039226, International Patent Publication No. WO2019/039341, International Patent Publication No. WO2019/039186, International Patent Publication No. WO2019/044479, Japanese Unexamined Patent Application Publication No. 2019-44158, or International Patent Publication No. WO2019/163282.

Meanwhile, examples of a commercially available fluorine-containing compound include the KY-100 series (KY-178, KY-185, KY-195, etc.) manufactured by Shin-Etsu Chemical Co., Ltd., Afluid (registered trademark) S550 manufactured by AGC Inc., Optool (registered trademark) DSX, Optool (registered trademark) AES, Optool (registered trademark) UF503, or Optool (registered trademark) UD509 manufactured by Daikin Industries, Ltd.

The proportion of the present compound in the present composition is lower than 100 mass %, preferably no lower than 60 mass %, more preferably no lower than 70 mass %, or even more preferably no lower than 80 mass %.

In a case where the present composition includes another fluorine-containing compound, the proportion of the other fluorine-containing compound with respect to the present compound and the other fluorine-containing compound combined in the present composition is preferably no higher than 40 mass %, more preferably no higher than 30 mass %, or even more preferably no higher than 20 mass %.

The proportion of the present compound and the other fluorine-containing compound combined in the present composition is preferably no lower than 80 mass % or more preferably no lower than 85 mass %.

When the content of the present compound and the content of the other fluorine-containing compound are within the above ranges, a surface layer excels in water repellency or oil repellency, friction resistance, fingerprint stain removability, lubricity, or appearance.

### [Coating Liquid]

A coating liquid according to the present invention (also referred to below as the present coating liquid) includes the present compound or the present composition and a liquid medium. It suffices that the present coating liquid be a liquid, and the present coating liquid may be a solution or a dispersion.

It suffices that the present coating liquid include the present compound or the present composition, and the present coating liquid may include an impurity, such as a by-product produced in the process of manufacturing the present compound.

The concentration of the present compound or the present composition in the present coating liquid is preferably from 0.001 mass % to 40 mass %, more preferably from 0.01 mass % to 20 mass %, or even more preferably from 0.1 mass % to 10 mass %.

The liquid medium is preferably an organic solvent. An organic solvent may be a fluorine-based organic solvent or a non-fluorine-based organic solvent or may include both of such solvents.

Examples of a fluorine-based organic solvent include fluorinated alkane, a fluorinated aromatic compound, fluoroalkylether, fluorinated alkylamine, or fluoroalcohol.

For fluorinated alkane, a compound having a carbon number of from 4 to 8 is preferable. Examples of commercially available products include C₆F₁₃H (manufactured by AGC Inc., Asahiklin (registered trademark) AC-2000), C₆F1₃C2H₅ (manufactured by AGC Inc., Asahiklin (registered trademark) AC-6000), or C₂F₅CHFCHFCF₃ (manufactured by The Chemours Company, Vertrel (registered trademark) XF).

Examples of a fluorinated aromatic compound include hexafluorobenzene, trifluoromethylbenzene, perfluorotoluene, or bis(trifluoromethyl)benzene.

For fluoroalkylether, a compound having a carbon number of from 4 to 12 is preferable. Examples of commercially available products include CF₃CH₂OCF₂CF₂H (manufactured by AGC Inc., Asahiklin (registered trademark) AE-3000), C₄F9OCH₃ (manufactured by 3M, Novec (registered trademark) 7100), C₄F₉OC₂H₅ (manufactured by 3M, Novec (registered trademark) 7200), or C₂F₅CF(OCH₃)C₃F₇ (manufactured by 3M, Novec (registered trademark) 7300).

Examples of fluorinated alkylamine include perfluorotripropylamine or perfluorotributylamine.

Examples of fluoroalcohol include 2,2,3,3-tetrafluoropropanol, 2,2,2-trifluoroethanol, or hexafluoroisopropanol.

For a non-fluorine-based organic solvent, a compound consisting of a hydrogen atom and an oxygen atom or a compound consisting of a hydrogen atom, a carbon atom, and an oxygen atom is preferable, and examples of such include a hydrocarbon-based organic solvent, an alcohol-based organic solvent, a ketone-based organic solvent, an ether-based organic solvent, or an ester-based organic solvent.

The present coating liquid includes a liquid medium preferably in the range of from 75 mass % to 99.999 mass %, more preferably in the range of from 85 mass % to 99.99 mass %, or particularly preferably in the range of from 90 mass % to 99.9 mass %.

The present coating liquid may include, aside from the present compound or the present composition and a liquid medium, another component within a range that does not compromise the advantageous effects of the present invention.

Examples of such a component include a known additive, such as an acid catalyst or a basic catalyst, that promotes hydrolysis and a condensation reaction of a hydrolytic silyl group.

The content of another component in the present coating liquid is preferably no higher than 10 mass % or more preferably no higher than 1 mass %.

The concentration of the present compound and another component combined or the concentration of the present composition and another component combined in the present coating liquid (also referred to below as a solid content concentration) is preferably from 0.001 mass % to 40 mass %, more preferably from 0.01 mass % to 20 mass %, even more preferably from 0.01 mass % to 10 mass %, or particularly preferably from 0.01 mass % to 1 mass %. The solid content concentration of a coating liquid is a value calculated from the mass of the coating liquid held before heating and the mass held after the coating liquid has been heated for 4 hours in a convection-current dryer of 120° C.

### [Article]

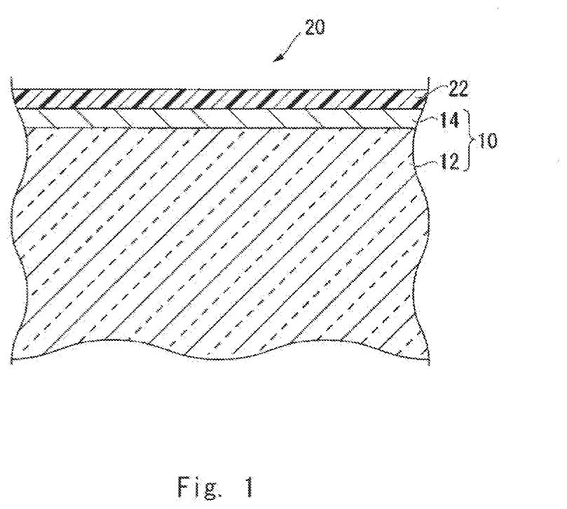

FIG. 1 is a schematic sectional view illustrating an example of an article according to the present invention. A first article according to the present invention is an article 20 that includes a substrate 12, a base layer 14, and a surface layer 22 in this order.

The base layer contains an oxide including silicon, and the surface layer contains a condensate of the present compound.

The material and the shape of the substrate in the first article described above may be selected as appropriate in accordance with the intended use or the like of the present article. Examples of the material for the substrate include glass, resin, sapphire, gold, ceramics, stone, or a composite material of the above. Glass may be chemically strengthened. In particular, examples of a substrate desired to have water repellency or oil repellency include a substrate for a touch panel, a substrate for a display, or a material forming a housing of an electronic device. A substrate for a touch panel or a substrate for a display is light-transmissive. Being “light-transmissive” means that such a substrate has a normal incidence visible light transmittance as set forth by JIS R3106:1998 (ISO 9050:1990) of 25% or higher. Preferred materials for a substrate for a touch panel include glass or transparent resin.

The surface of the substrate where the base layer is to be provided may be subjected to surface treatment, such as corona discharge treatment, plasma treatment, or plasma graft polymerization treatment. The surface subjected to surface treatment further excels in adhesiveness between the substrate and the base layer, and as a result, the abrasion resistance of the surface layer further improves. From the standpoint of the surface layer further excelling in abrasion resistance, the surface treatment is preferably corona discharge treatment or plasma treatment.

The base layer is a layer that contains an oxide including at least silicon and may further include another element. As the base layer contains silicon oxide, a partial structure (2) of the present compound undergoes dehydration condensation, and a Si—O—Si bond is formed between the base layer and the surface layer, and the surface layer that excels in abrasion durability is formed.

The content of silicon oxide in the base layer may be no lower than 65 mass % or is preferably no lower than 80 mass %, more preferably no lower than 85 mass %, or even more preferably no lower than 90 mass %. When the content of silicon oxide is no lower than the lower limit value of the aforementioned ranges, a Si—O—Si bond is formed sufficiently in the base layer, and the mechanical properties of the base layer can be ensured sufficiently. The content of silicon oxide is the balance obtained by excluding the total content of other elements (the amount in an oxide-equivalent in the case of an oxide) from the mass of the base layer.

From the standpoint of durability of the surface layer, the oxide in the base layer preferably further contains one or more elements selected from an alkali metal element, an alkaline-earth metal element, a platinum group element, boron, aluminum, phosphorus, titanium, zirconium, iron, nickel, chromium, molybdenum, or tungsten. As the oxide contains any of these elements, the bond between the base layer and the present compound is enhanced, and the abrasion resistance improves.

In a case where the base layer includes one or more selected from iron, nickel, or chromium, the total content of these in terms of the proportion to silicon oxide is preferably from 10 mass ppm to 1,100 mass ppm, more preferably from 50 mass ppm to 1,100 mass ppm, even more preferably from 50 mass ppm to 500 mass ppm, or particularly preferably from 50 mass ppm to 250 mass ppm.

In a case where the base layer includes one or more selected from aluminum or zirconium, the total content of these is preferably from 10 mass ppm to 2,500 mass ppm, more preferably from 15 mass ppm to 2,000 mass ppm, or even more preferably from 20 mass ppm to 1,000 mass ppm.

In a case where the base layer includes an alkali metal element, the total content of such is preferably from 0.05 mass % to 15 mass %, more preferably from 0.1 mass % to 13 mass %, or even more preferably from 1.0 mass % to 10 mass %. Examples of an alkali metal element include lithium, sodium, potassium, rubidium, or cesium.

In a case where the base layer includes a platinum group element, the total content of such is preferably no lower than 0.02 mass ppm nor higher than 800 mass ppm, more preferably no lower than 0.04 mass ppm nor higher than 600 mass ppm, or even more preferably no lower than 0.7 mass ppm nor higher than 200 mass ppm. Examples of a platinum group element include platinum, rhodium, ruthenium, palladium, osmium, or iridium.

In a case where the base layer includes one or more selected from boron or phosphorus, from the standpoint of abrasion resistance of the surface layer, the total content of these in terms of the ratio of the molarity of the total of boron and phosphorus to the molarity of silicon is preferably from 0.003 to 9, more preferably from 0.003 to 2, or even more preferably from 0.003 to 0.5.

In a case where the base layer includes an alkaline-earth metal element, from the standpoint of abrasion resistance of the surface layer, the total content of such in terms of the ratio of the molarity of the total of the alkaline-earth metal element to the molarity of silicon is preferably from 0.005 to 5, more preferably from 0.005 to 2, or even more preferably from 0.007 to 2. Examples of an alkaline-earth metal element include lithium, sodium, potassium, lithium, or cesium.

From the standpoint of improving the adhesiveness of the present compound and improving the water repellency or oil repellency or the abrasion resistance of the article, the base layer is preferably a silicon oxide layer including an alkali metal atom. In particular, it is preferable that, in the silicon oxide layer, the mean value of the density of alkali metal atoms in a region that is from 0.1 nm to 0.3 nm deep from the surface of the base layer where the base layer makes contact with the surface layer be no lower than 2.0×10¹⁹ atoms/cm³. Meanwhile, from the standpoint of ensuring the mechanical properties of the silicon oxide layer sufficiently, it is preferable that the mean value of the density of the alkali metal atoms be no higher than 4.0×10²² atoms/cm³.

The thickness of the base layer is preferably from 1 nm to 200 nm or particularly preferably from 2 nm to 20 nm. When the thickness of the base layer is no smaller than the lower limit value of the above ranges, the effect of improving adhesiveness of the base layer is more likely to be obtained sufficiently. When the thickness of the base layer is no greater than the upper limit value of the above ranges, the abrasion resistance of the base layer itself increases. Examples of a method of measuring the thickness of the base layer include a method in which the section of the base layer is observed with an electron microscope (SEM, TEM, etc.) or a method in which an optical coherence membrane thickness gauge, a spectroscopic ellipsometer, a profilograph, or the like is used.

Examples of a method of forming the base layer include a method in which a deposition material having a constitution of a desired base layer is deposited on a surface of a substrate.

One example of a deposition method is a vacuum deposition method. In a vacuum deposition method, a deposition material is vaporized in a vacuum tank and is adhered to a surface of a substrate.

The temperature to be held during deposition (e.g., when a vacuum deposition device is used, the temperature of the boat in which the deposition material is placed) is preferably from 100° C. to 3,000° C. or particularly preferably from 500° C. to 3,000° C.

The pressure to be held during deposition (e.g., when a vacuum deposition device is used, the absolute pressure inside the tank where the deposition material is placed) is preferably no higher than 1 Pa or particularly preferably no higher than 0.1 Pa.

In a case where the base layer is formed with a deposition material, one deposition material may be used, or two or more deposition materials including different elements may be used.

Examples of a method of vaporizing a deposition material include a resistance heating method in which a deposition material is molten on a resistance heating boat made of a high-melting-point metal and thus vaporized, or an electron gun method in which a deposition material is irradiated with an electron beam to directly heat the deposition material so as to melt its surface and thus vaporized. From the standpoint of being able to locally heat a deposition material to vaporize even a high-melting-point substance or from the standpoint of being able to keep the temperature of a portion not irradiated with an electron beam low so as to prevent a reaction with a container or mixing in of an impurity, the method of vaporizing a deposition material is preferably an electron gun method. A deposition material used in an electron gun method is preferably a molten granular body or a sintered body, as such bodies are less likely to splash even when an airflow is produced.

The surface layer on the base layer contains a condensate of the present compound. The condensate of the present compound includes a condensate in which a hydrolytic silyl group in the present compound has undergone a hydrolysis reaction to form a silanol group (Si—OH) and the silanol group has undergone a condensation reaction between molecules to form a Si—O—Si bond, or a condensate in which a silanol group in the present compound has undergone a condensation reaction with a silanol group or a Si-OM group (in which M is an alkali metal element) on the surface of the base layer to form a Si—O—Si bond. The surface layer may include a condensate of a fluorine-containing compound other than the present compound. In other words, the surface layer includes a fluorine-containing compound having a reactive silyl group in a state in which a part or the whole of the reactive silyl group of the fluorine-containing compound has undergone a condensation reaction.

The surface layer has a thickness of preferably from 1 nm to 100 nm or particularly preferably from 1 nm to 50 nm. When the thickness of the surface layer is no smaller than the lower limit value of the above ranges, the effect provided by the surface layer is obtained sufficiently. When the thickness of the surface layer is no greater than the upper limit value of the above ranges, the utilization efficiency is high.

The thickness of the surface layer is a thickness obtained with an X-ray diffractometer for thin film analysis. The thickness of the surface layer can be calculated by a vibration cycle of an interference pattern of reflected X-rays obtained through an X-ray reflectance technique by use of an X-ray diffractometer for thin film analysis.

A second article according to the present invention is an article that includes a substrate 12 and a surface layer 22 in this order.

The substrate contains an oxide including silicon.

The surface layer contains a condensate of the present compound.

The substrate in the second article has a constitution of the base layer of the first article described above, and thus the second article excels in abrasion durability of the surface layer even when the surface layer is formed directly on the substrate.

It suffices that a material for the substrate of the second article have a constitution of the base layer described above, and the substrate may be, for example, a glass substrate. Specifics of the material for the substrate are similar to those of the material for the base layer described above, and thus description thereof will be omitted here. In addition, the configuration of the surface layer is similar to that of the first article, and thus description thereof will be omitted here.

### [Method of Manufacturing Article]

A method of manufacturing an article according to the present invention is a method of forming a surface layer through a dry coating technique or a wet coating technique with use of the fluorine-containing compound, the composition containing the fluorine-containing compound, or the coating liquid.

The present compound and the present composition can be used unmodified in a dry coating technique. The present compound and the present composition are suitable for forming a surface layer that excels in adhesiveness through a dry coating technique. Examples of a dry coating technique include vacuum deposition, CVD, or sputtering. From the standpoint of suppressing decomposition of the present compound or the simplicity of a device, a vacuum deposition method can be used favorably.

In vacuum deposition, a pellet-form substance in which the present compound is supported in a porous metal body made of a metal material, such as iron or steel, may be used. The pellet-form substance in which the present compound is supported can be manufactured by impregnating a porous metal body with a solution of the present compound and drying the resultant to remove the liquid medium. As a solution of the present compound, the present coating liquid can be used.

The present coating liquid can be used favorably in a wet coating technique. Examples of a wet coating technique include a spin coating technique, a wipe coating technique, a spray coating technique, a squeegee coating technique, a dip coating technique, a die coating technique, an inkjet technique, a flow coating technique, a roll coating technique, a casting technique, a Langmuir-Blodgett technique, or a gravure coating technique.

In order to improve friction resistance of the surface layer, an operation for accelerating a reaction between the present compound and a substrate may be performed as necessary. Examples of such an operation include heating, humidifying, or light irradiation. For example, by heating a substrate on which a surface layer is formed in atmosphere with moisture, a reaction such as a hydrolysis reaction with a hydrolytic group, a reaction between a hydroxyl group on the surface of the substrate and a silanol group, or production of a siloxane bond through a condensation reaction of a silanol group can be accelerated.

After surface treatment, a compound that is in the surface layer and that has not chemically bonded with another compound or the substrate may be removed as necessary. Examples of a specific method for such include a method in which a solvent is allowed to flow on the surface layer or a method in which the surface layer is wiped with a cloth soaked with a solvent.

The present invention will be described in further detail through examples below, but the present invention is not limited by these examples. In the following, “%” means “mass %” unless specifically indicated otherwise. Examples 1 to 4 and 8 to 11 are the examples, and Examples 5 to 7 and 12 to 14 are comparative example.

### Synthesis Example 1: Synthesis of Compound (1)

Diethyldiallylmalonate (60.0 g), lithium chloride (23.7 g), water (6.45 g), and dimethyl sulfoxide (263 g) were added and stirred at 160° C. The resultant was cooled to room temperature, added with water, and extracted with ethyl acetate. Hexane was added to the organic phase, and the resultant was washed with a saturated saline solution and dried with sodium sulfate. The resultant was filtered to distill the solvent, and thus 39.5 g of Compound (1) below was obtained.

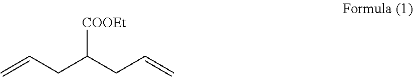

The NMR spectrum of Compound (1);

¹H-NMR (400 MHz, Chloroform-d) δ (ppm): (ddt, J=17.1, 10.1, 7.0 Hz, 2H), from 5.06 to 4.94 (m, 4H), 4.09 (q, J=7.1 Hz, 2H), 2.47 (ddd, J=14.0, 8.0, 6.1 Hz, 1H), 2.33 (dt, J=14.9, 7.5 Hz, 2H), 2.22 (dt, J=14.1, 6.5 Hz, 2H), 1.21 (t, J=7.1 Hz, 3H).

### Synthesis Example 2: Synthesis of Compound (2)

THF (260 mL) and diisopropylamine (41.6 mL) were added, and then the solution was cooled to −78° C. An n-butyllithium hexane solution (2.76 M, 96.6 mL) was added, and the temperature of the resultant was raised to 0° C. The above was stirred and then cooled to −78° C., and a THF solution of lithium diisopropylamide (LDA) was prepared. Compound (1) above (39.5 g) was added to the THF solution and stirred. Then, allyl bromide (24.1 mL) was added. The temperature of the resultant was raised to 0° C., and 1M hydrochloric acid was added to distill THF under reduced pressure. The above was extracted with dichloromethane, and then sodium sulfate was added. The resultant was filtered to distill the solvent, and flash column chromatography involving silica gel was carried out to obtain 45.0 g of Compound (2) below.

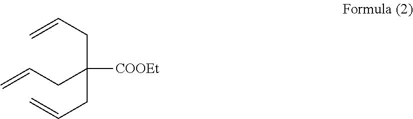

The NMR spectrum of Compound (2);

¹H-NMR (400 MHz, Chloroform-d) δ (ppm): from 5.74 to 5.62 (m, 3H), 5.04 (dd, J=13.6, 1.9 Hz, 6H), 4.10 (q, J=7.1 Hz, 2H), 2.29 (d, J=7.4 Hz, 6H), 1.22 (t, J=7.1 Hz, 3H).

### Synthesis Example 3: Synthesis of Compound (3)

Compound (2) above (45.0 g) was dissolved in THF, and the resultant was cooled to 0° C. A THF solution of lithium aluminum hydride (104 mL, 260 mmol) was added and stirred. Water and a 15% sodium hydroxide solution were added and stirred at room temperature. Then, the resultant was diluted with dichloromethane. The above was filtered to distill the solvent, and flash column chromatography involving silica gel was carried out to obtain 31.3 g of Compound (3) below.

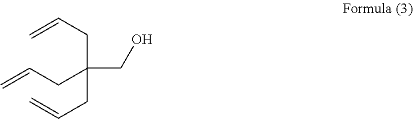

The NMR spectrum of Compound (3);

¹H-NMR (400 MHz, Chloroform-d) δ (ppm): from 5.90 to 5.76 (m, 3H), from 5.10 to 5.02 (m, 6H), 3.38 (s, 2H), 2.03 (dt, J=7.5, 1.2 Hz, 6H), 1.45 (s, 1H).

### Synthesis Example 4: Synthesis of Compound (4)

Compound (1) above (8.4 g) was dissolved in THF, and the resultant was cooled to 0° C. A THF solution of lithium aluminum hydride (21 mL, 52 mmol) was added and stirred. Water and a 15% sodium hydroxide solution were added and stirred at room temperature. Then, the resultant was diluted with dichloromethane. The above was filtered to distill the solvent, and flash column chromatography involving silica gel was carried out to obtain 5.4 g of Compound (4) below.

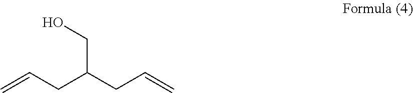

The NMR spectrum of Compound (4);

¹H-NMR (400 MHz, Chloroform-d) δ (ppm): 5.79 (m, 2H), from 5.07 to 4.97 (m, 4H), 3.55 (t, J=5.5 Hz, 2H), 2.10 (m, 4H), from 1.76 to 1.64 (m, 1H).

### Synthesis Example 5: Synthesis of Compound (5)

Compound (4) above (2.5 g) was dissolved in dichloromethane, and the resultant was cooled to 0° C. The Dess-Martin reagent (10 g) was added and stirred. The above was filtered to distill the solvent, and flash column chromatography involving silica gel was carried out to obtain 2.1 g of Compound (5) below.

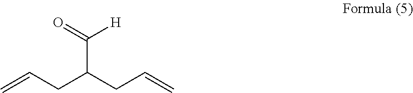

The NMR spectrum of Compound (5);

¹H-NMR (400 MHz, Chloroform-d) δ (ppm): 9.65 (d, J=1.9 Hz, 1H), 5.74 (m, 2H), from 5.16 to 5.02 (m, 4H), from 2.53 to 2.34 (m, 3H), from 2.32 to 2.19 (m, 2H).

### Synthesis Example 6: Synthesis of Compound (6)

Compound (5) above (2.1 g) was dissolved in THF, and the resultant was cooled to 0° C. An ethyl ether solution of allylmagnesiumbromide (30 mL, 21 mmol) was added and stirred. 1M hydrochloric acid was added to distill THF under reduced pressure. The resultant was extracted with dichloromethane, and then sodium sulfate was added. The above was filtered to distill the solvent, and flash column chromatography involving silica gel was carried out to obtain 1.8 g of Compound (6) below.

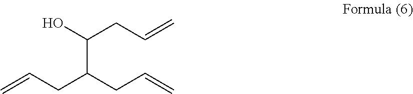

The NMR spectrum of Compound (6);

¹H-NMR (400 MHz, Chloroform-d) δ (ppm): from 5.89 to 5.72 (m, 3H), from 5.18 to 4.98 (m, 6H), 3.66 (s, 1H), from 2.37 to 2.01 (m, 6H), from 1.71 to 1.57 (m, 1H).

### Synthesis Example 7: Synthesis of Compound (1-1)

Compound (1-1) below was obtained in accordance with the method described in Example 6 of International Publication No. WO2013/121984.

CF₃—O—(CF₂CF₂O—CF₂CF₂CF₂CF₂O)ₓ₁(CF₂CF₂O)—CF₂CF₂CF₂—C(O)OCH₃ Formula (1-1)

Mean value of the number of units x1: 14

### Synthesis Example 8: Synthesis of Compounds (1-2) and (1-3)

1,3-bistrifluoromethylbenzene (10 g) was added to Compound (1-1) above (10.0 g), and then triethylsilane (450 mg) and trispentafluorophenylborane (10 mg) were added. The resultant was stirred for 2 hours at room temperature, and then a low-boiling-point component was distilled to obtain 10.3 g of a mixture of Compounds (1-2) and (1-3) below.

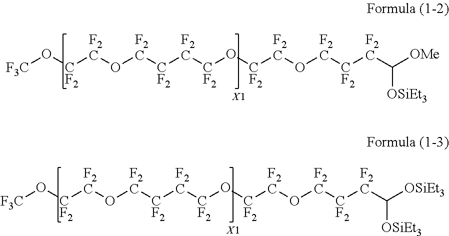

Mean value of the number of units x1: 14

The NMR spectrum of Compound (1-2);

¹H-NMR (400 MHz, Chloroform-d) δ (ppm): from 5.12 to 5.02 (1H), 3.46 (s, 3H), from 1.02 to 0.90 (9H), from 0.75 to 0.58 (6H).

¹⁹F-NMR (376 MHz, Chloroform-d) δ (ppm): −54.85, −82.32, −86.23, −87.56, −89.70, −124.15, −124.73.

The NMR spectrum of Compound (1−3);

¹H-NMR (400 MHz, Chloroform-d) δ (ppm): from 5.57 to 5.52 (1H), from 1.02 to 0.90 (18H), from 0.75 to 0.58 (12H).

¹⁹F-NMR (376 MHz, Chloroform-d) δ (ppm): −54.85, −82.32, −86.23, −87.56, −89.70, −124.15, −124.73.

### Synthesis Example 9: Synthesis of Compounds (1−4) and (1−5)

1,3-bistrifluoromethylbenzene (10 g) was added to a mixture (5.0 g) of (1−2) and (1−3) above, and then methanol (5.0 g) and concentrated sulfuric acid (2.5 g) were added. The resultant was stirred for 3 hours at 60° C. and then extracted with AC-6000. The solvent was removed to obtain 4.9 g of a mixture of Compounds (1−4) and (1−5) below.

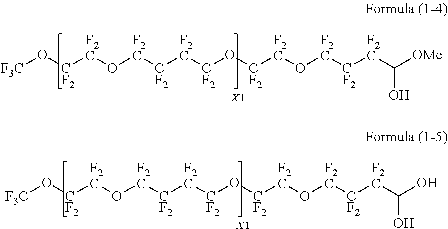

Mean value of the number of units x1: 14

The NMR spectrum of Compound (1−4);

¹H-NMR (400 MHz, Chloroform-d) δ (ppm): from 4.89 to 4.73 (1H), 3.45 (s, 3H).

¹⁹F-NMR (376 MHz, Chloroform-d) δ (ppm): −54.84, −82.31, −86.22, −87.54, −89.69, −124.70, −125.25, −128.63.

The NMR spectrum of Compound (1−5);

¹H-NMR (400 MHz, Chloroform-d) δ (ppm): from 5.34 to 5.21 (1H).

¹⁹F-NMR (376 MHz, Chloroform-d) δ (ppm): −54.84, −82.31, −86.22, −87.54, −89.69, −124.70, −125.25, −128.63.

### Synthesis Example 10: Synthesis of Compound (1−6)

A mixture (4.9 g) of (1−4) and (1−5) above were loaded into a vacuum drying oven to undergo vacuum concentration for 16 hours at 100° C., and thus 4.8 g of Compound (1−6) below was obtained.

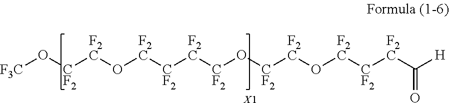

Mean value of the number of units x1: 14

The NMR spectrum of Compound (1−6);

¹H-NMR (400 MHz, Chloroform-d) δ (ppm): 9.42 (t, J=3.3 Hz, 1H).

¹⁹F-NMR (376 MHz, Chloroform-d) δ (ppm): −54.85, −82.32, −86.24, −87.56, −89.68, −124.73, −125.27, −126.50.

### Synthesis Example 11: Synthesis of Compound (1−7)

1,3-bistrifluoromethylbenzene (1.0 g) was added to Compound (1−6) above (1.0 g), and then Compound (3) above (60 mg) was added. The resultant was stirred for 1 hour at room temperature, and then bis(2-methoxyethyl)aminosulfur=trifluoride (300 mg) was added. The resultant was heated to 80° C. and stirred for 1 hour, and then methanol was added to extract with AC-6000. The solvent was distilled, and flash column chromatography involving silica gel was carried out to obtain 0.85 g of Compound (1−7) below.

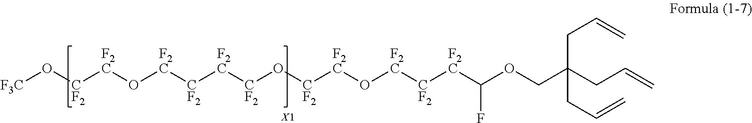

Mean value of the number of units x1: 14

The NMR spectrum of Compound (1−7);

¹H-NMR (400 MHz, Chloroform-d) δ (ppm): from 5.83 to 5.67 (3H), from 5.56 to 5.30 (1H), from 5.14 to 4.96 (6H), from 3.79 to 3.69 (1H), from 3.43 to 3.34 (1H), from 2.07 to 1.96 (6H).

¹⁹F-NMR (376 MHz, Chloroform-d) δ (ppm): −54.85, −82.32, −86.23, −87.56, −89.70, −124.73, −125.0, −125.21, −125.43, −125.98, −126.15, from −141.30 to −141.80.

### Example 1: Synthesis of Fluorine-Containing Ether Compound (1−8)

AC-6000 (1.0 g), Compound (1−7) above (0.5 g), a toluene solution of platinum/1,3-divinyl-1,1,3,3-tetramethyldisiloxane complex (platinum content 3%, 8.3 mg), aniline (2.6 mg), and trimethoxysilane (125 mg) were added and stirred at 40° C., and then the solvent was distilled under reduced pressure to obtain 0.51 g of a fluorine-containing ether compound (1−8) below.

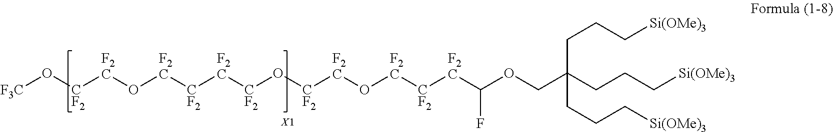

Mean value of the number of units x1: 14

The NMR spectrum of Compound (1−8);

¹H-NMR (400 MHz, Chloroform-d) δ (ppm): from 5.61 to 5.25 (1H), from 3.80 to 3.68 (1H), from 3.64 to 3.45 (27H), from 3.44 to 3.31 (1H), from 1.57 to 1.29 (12H), from 0.75 to 0.47 (6H). ¹⁹F-NMR (376 MHz, Chloroform-d) δ (ppm): −54.85, −82.32, −86.23, −87.56, −89.70, −124.73, −125.0, −125.21, −125.43, −125.98, −126.15, from −141.30 to −141.80.

### Synthesis Example 12: Synthesis of Compound (2−1)

1,3-bistrifluoromethylbenzene (1.0 g) was added to Compound (1−6) above (1.0 g), and then 1,6-heptadien-4-ol (40 mg) was added. The resultant was stirred for 5 hours at room temperature, and then bis(2-methoxyethyl)aminosulfur=trifluoride (300 mg) was added. The resultant was heated to 80° C. and stirred for 1 hour, and then methanol was added to extract with AC-6000. The solvent was distilled, and flash column chromatography involving silica gel was carried out to obtain 0.65 g of Compound (2−1) below.

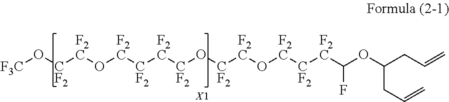

Mean value of the number of units x1: 14

The NMR spectrum of Compound (2−1);

¹H-NMR (400 MHz, Chloroform-d) δ (ppm): from 5.85 to 5.50 (3H), from 5.13 to 4.93 (4H), from 3.88 to 3.78 (1H), from 2.42 to 2.19 (4H).

¹⁹F-NMR (376 MHz, Chloroform-d) δ (ppm): −54.85, −82.33, −86.24, −87.57, −89.71, −124.74, −124.94, −125.18, from −136.79 to −137.11.

### Example 2: Synthesis of Fluorine-Containing Ether Compound (2−2)

AC-6000 (1.0 g), Compound (2−1) above (0.5 g), a toluene solution of platinum/1,3-divinyl-1,1,3,3-tetramethyldisiloxane complex (platinum content 3%, 8.3 mg), aniline (2.6 mg), and trimethoxysilane (83 mg) were added and stirred at 40° C., and then the solvent was distilled under reduced pressure to obtain 0.5 g of a fluorine-containing ether compound (2−2) below.

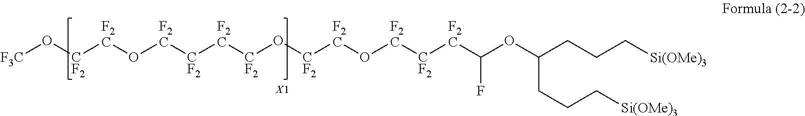

Mean value of the number of units x1: 14

The NMR spectrum of Compound (2−2);

¹H-NMR (400 MHz, Chloroform-d) δ (ppm): from 5.77 to 5.51 (1H), from 3.98 to 3.75 (1H), from 3.65 to 3.43 (18H), from 1.78 to 1.49 (8H), from 0.88 to 0.71 (4H).

¹⁹F-NMR (376 MHz, Chloroform-d) δ (ppm): −54.85, −82.33, −86.24, −87.57, −89.71, −124.74, −124.94, −125.18, from −136.79 to −137.11.

### Synthesis Example 13: Synthesis of Compound (3−1)

1,3-bistrifluoromethylbenzene (1.0 g) was added to Compound (1−6) above (1.0 g), and then Compound (6) above (60 mg) was added. The resultant was stirred for 72 hours at room temperature, and then bis(2-methoxyethyl)aminosulfur=trifluoride (300 mg) was added. The resultant was heated to 80° C. and stirred for 1 hour, and then methanol was added to extract with AC-6000. The solvent was distilled, and flash column chromatography involving silica gel was carried out to obtain 0.55 g of Compound (3−1) below.

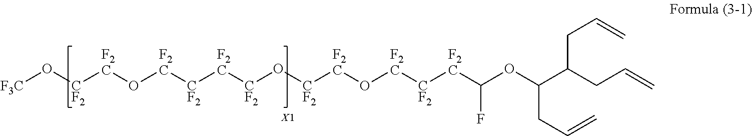

Mean value of the number of units x1: 14

The NMR spectrum of Compound (3−1);

¹H-NMR (400 MHz, Chloroform-d) δ (ppm): from 5.77 to 5.33 (4H), from 5.08 to 4.62 (6H), from 3.93 to 3.66 (1H), from 2.36 to 1.46 (7H).

¹⁹F-NMR (376 MHz, Chloroform-d) δ (ppm): −54.85, −82.34, −86.24, −87.57, −89.70, −124.75, −125.26, from −133.46 to −133.84, from −136.50 to −136.89.

### Example 3: Synthesis of Fluorine-Containing Ether Compound (3−2)

AC-6000 (1.0 g), Compound (3−1) above (0.5 g), a toluene solution of platinum/1,3-divinyl-1,1,3,3-tetramethyldisiloxane complex (platinum content 3%, 8.3 mg), aniline (2.6 mg), and trimethoxysilane (125 mg) were added and stirred at 40° C., and then the solvent was distilled under reduced pressure to obtain 0.51 g of a fluorine-containing ether compound (3−2) below.

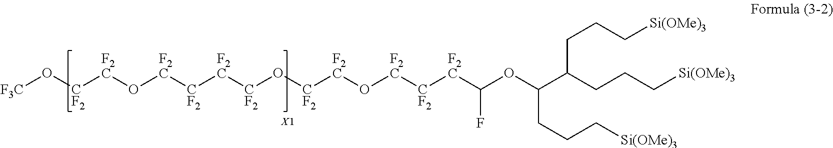

Mean value of the number of units x1: 14

The NMR spectrum of Compound (3−2);

¹H-NMR (400 MHz, Chloroform-d) (ppm): from 5.80 to 5.50 (1H), from 4.01 to 3.86 (1H), from 3.78 to 3.30 (27H), from 1.86 to 1.37 (13H), from 0.85 to 0.45 (6H).

¹⁹F-NMR (376 MHz, Chloroform-d) δ (ppm): −54.85, −82.34, −86.24, −87.57, −89.70, −124.75, −125.26, from −133.46 to −133.84, from −136.50 to −136.89.

### Synthesis Example 14: Synthesis of Compound (4−1)

1,3-bistrifluoromethylbenzene (1.0 g) was added to Compound (1−6) above (1.0 g), and then allyl alcohol (20 mg) was added. The resultant was stirred for 1 hour at room temperature, and then bis(2-methoxyethyl)aminosulfur=trifluoride (300 mg) was added. The resultant was heated to 80° C. and stirred for 1 hour, and then methanol was added to extract with AC-6000. The solvent was distilled, and flash column chromatography involving silica gel was carried out to obtain 0.77 g of Compound (4−1) below.

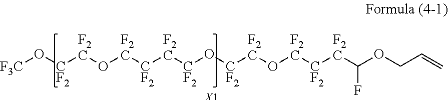

The NMR spectrum of Compound (4−1);

¹H-NMR (400 MHz, Chloroform-d) δ (ppm): from 5.90 to 5.73 (1H), from 5.68 to 5.43 (1H), from 5.35 to 5.13 (2H), from 4.41 to 4.13 (2H)

¹⁹F-NMR (376 MHz, Chloroform-d) δ (ppm): −54.85, −82.34, −86.24, −87.57, −89.70, −124.67, from −125.14 to −125.29, −126.16, from −142.64 to −143.30

### Example 4: Synthesis of Fluorine-Containing Ether Compound (4−2)

AC-6000 (1.0 g), Compound (4−1) above (0.5 g), a toluene solution of platinum/1,3-divinyl-1,1,3,3-tetramethyldisiloxane complex (platinum content 3%, 8.0 mg), aniline (2.8 mg), and trimethoxysilane (50 mg) were added and stirred at 40° C., and then the solvent was distilled under reduced pressure to obtain 0.51 g of a fluorine-containing ether compound (4−2) below.

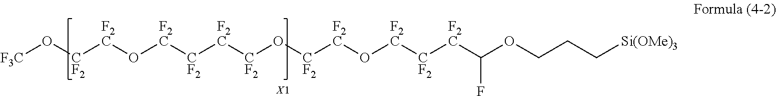

The NMR spectrum of Compound (4−2);

¹H-NMR (400 MHz, Chloroform-d) δ (ppm): from 5.65 to 5.36 (1H), from 3.77 to 3.39 (13H), from 1.87 to 1.71 (2H), from 0.76 to 0.63 (2H)

¹⁹F-NMR (376 MHz, Chloroform-d) δ (ppm): −54.85, −82.34, −86.24, −87.57, −89.70, −124.67, from −125.14 to −125.29, −126.16, from −142.64 to −143.30

### Example 5: Synthesis of Compound (5−1)

Compound (5−1) below was obtained in accordance with the method described in Synthesis Examples 1 to 3 of International Publication No. WO2017/022437.

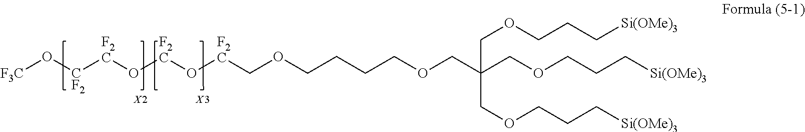

The mean value of the number of repeating units x2 is 21, and the mean value of the number of repeating units x3 is 19.

### Example 6: Synthesis of Compound (6−1)

Compound (6−1) below was obtained in accordance with the method described in Example 5 of International Publication No. WO2017/038830.

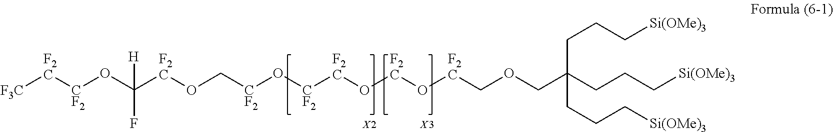

The mean value of the number of repeating units x2 is 21, and the mean value of the number of repeating units x3 is 19.

### Synthesis of Compound (7−1)

Compound (7−1) below was obtained in accordance with the method described in Example 7 of International Publication No. WO2013/121984.

CF₃—O—(CF₂CF₂O—CF₂CF₂CF₂CF₂O)ₓ₁(CF₂CF₂O)—CF₂CF₂CF₂—CH₂OH Formula (7−1)

Mean value of the number of units x1: 14

### Synthesis of Compound (7−2)

1,3-bistrifluoromethylbenzene (1.0 g) was added to Compound (7−1) above (1.0 g), and then cesium carbonate (5.0 g) and allyl bromide (200 mg) were added. The resultant was stirred overnight at 60° C. and then added with hydrochloric acid to be extracted. The solvent was distilled, and flash column chromatography involving silica gel was carried out to obtain 0.83 g of Compound (7−2) below.

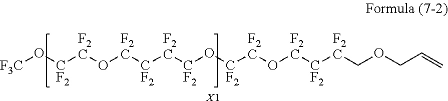

The NMR spectrum of Compound (7−2);

¹H-NMR (400 MHz, Chloroform-d) δ (ppm): from 5.85 to 5.69 (1H), from 5.29 to 5.04 (2H), from 4.08 to 3.98 (2H), from 3.94 to 3.81 (2H)

¹⁹F-NMR (376 MHz, Chloroform-d) δ (ppm): −54.85, −82.34, −86.24, −87.57, −89.70, −118.86, −124.67, −125.97

### Example 7: Synthesis of Compound (7−3)

AC-6000 (1.0 g), Compound (7−2) above (0.5 g), a toluene solution of platinum/1,3-divinyl-1,1,3,3-tetramethyldisiloxane complex (platinum content 3%, 8.0 mg), aniline (2.8 mg), and trimethoxysilane (50 mg) were added and stirred at 40° C., and then the solvent was distilled under reduced pressure to obtain 0.51 g of a fluorine-containing ether compound (7−3) below.

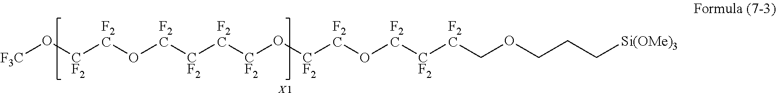

The NMR spectrum of Compound (7−3);

¹H-NMR (400 MHz, Chloroform-d) δ (ppm): from 3.98 to 3.81 (2H), from 3.65 to 3.47 (11H), from 1.79 to 1.63 (2H), from 0.73 to 0.59 (2H)

¹⁹F-NMR (376 MHz, Chloroform-d) δ (ppm): −54.85, −82.34, −86.24, −87.57, −89.70, −118.86, −124.67, −125.97

### Examples 8 to 14: Manufacturing and Evaluation of Article

Substrates were subjected to surface treatment with use of the respective compounds obtained in Examples 1 to 7, and articles of Examples 8 to 14 were obtained. For surface treatment, the dry coating technique or the wet coating technique described below was used in the examples. Chemically strengthened glass was used for the substrates. Obtained articles were evaluated through the following method. The result is shown in Table 1.

### (Dry Coating Technique)

Dry coating was done with use of a vacuum deposition device (manufactured by ULVAC, Inc., VTR350M) (vacuum deposition technique). A molybdenum boat inside the vacuum deposition device was charged with 0.5 g of each compound, and the inside of the vacuum deposition device was exhausted to a pressure of no higher than 1×10⁻³ Pa. The boat having a compound placed thereon was heated at a heating rate of no higher than 10° C./min, and the shutter was opened when the rate of deposition exceeded 1 nm/sec as measured by a quartz-crystal oscillator thickness gauge to start film formation on a surface of a substrate. The shutter was closed when the film thickness reached about 50 nm to end the film formation on the surface of the substrate. The substrate on which the compound was deposited was subjected to heat treatment for 30 minutes at 200° C. and washed with dichloropentafluoropropane (manufactured by AGC Inc., AK-225), and thus an article having a surface layer on the surface of the substrate was obtained.

### Method of Evaluation

### <Method of Measuring Contact Angle>

The contact angle of distilled water or n-hexadecane of about 2 μL placed on the surface of each surface layer was measured with use of a contact angle measuring device (manufactured by Kyowa Interface Science Co., Ltd., DM-500). The contact angle was measured at five different positions on the surface of each surface layer, and their mean value was calculated. The 2θ method was used to calculate each contact angle.

### <Initial Contact Angle>

For each surface layer, the initial water contact angle and the initial n-hexadecane contact angle were measured through the measurement method described above. The evaluation criteria are as follows.

Initial water contact angle:

A (good): 115 degrees or greater

C (unacceptable): smaller than 115 degrees

### <Friction Resistance (Steel Wool)>

Steel wool BONSTAR (#0000) was reciprocated on each surface layer 10,000 times at a pressure of 98.07 kPa and at a rate of 320 cm/min with use of a reciprocating traverse testing machine (manufactured by KNT Co., Ltd.) in accordance with JIS L0849:2013 (ISO 105-X12:2001), and then the water contact angle was measured through the method described above. The smaller the decrease in water repellency (water contact angle) after friction, the smaller the decrease in performance caused by the friction and the higher the friction resistance. The evaluation criteria are as follows.

A (excellent): The change in the water contact angle observed after reciprocation of 10,000 times is no greater than 4 degrees.

B (good): The change in the water contact angle observed after reciprocation of 10,000 times is greater than 4 degrees but no greater than 8 degrees.

C (unacceptable): The change in the water contact angle observed after reciprocation of 10,000 times is greater than 8 degrees.

### <Light Resistance>

Each surface layer was irradiated with light rays (650 W/m², from 300 to 700 nm) for 500 hours at a black panel temperature of 63° C. with use of a tabletop xenon arc lamp accelerated light resistance testing machine (product name: SUNTEST XLS+, manufactured by Toyo Seiki Seisaku-sho, Ltd.), and then the water contact angle was measured through the method described above. The evaluation criteria are as follows.

A (excellent): The change in the water contact angle observed after the accelerated light resistance testing is no greater than 5 degrees.

B (good): The change in the water contact angle observed after the accelerated light resistance testing is greater than 5 degrees but no greater than 10 degrees.

C (unacceptable): The change in the water contact angle observed after the accelerated light resistance testing is greater than 10.

TABLE 1

| | | Examples | | | | | | |
|---|---|---|---|---|---|---|---|---|
| | | 8 | 9 | 10 | 11 | 12 | 13 | 14 |
| Compounds | Types | Compound | Compound | Compound | Compound | Compound | Compound | Compound |
| | | (1-8) | (2-2) | (3-2) | (4-2) | (5-1) | (6-1) | (7-2) |
| Dry | Initial Contact | A | A | A | A | A | C | A |
| Coating | Angle \| Water | | | | | | | |
| | Friction Resistance | A | B | A | B | C | B | C |
| | Light Resistance | A | A | A | B | C | B | C |

As summarized in Table 1, the initial contact angle and the light resistance were good in the articles of Example 12 and Example 14 in which the compounds of Example 5 and Example 7, respectively, were used, but a decrease in the water contact angle was observed in particular after the friction testing. Meanwhile, in the article in which used was the compound of Example 6 in which the O—CHF group was located away from an adhesive group, the initial water repellency was insufficient.

In contrast, in the articles of Example 8 to Example 11 in which used were the fluorine-containing ether compounds of Example 1 to Example 4 having the O—CHF group at a linking part of a polyfluoropolyether chain and an adhesive group, a decrease in the water contact angle was suppressed both after the friction testing and after the light resistance testing, and they exhibited excellent durability.

An article having a surface layer including the present compound is used effectively as, for example but not limited to, an optical article, a touch panel, an anti-reflection film, anti-reflection glass, SiO₂ treatment glass, strengthened glass, sapphire glass, a quartz substrate, or a die metal to be used in a portion of a part of the following products.

Products: car navigation systems, mobile phones, digital cameras, digital video cameras, mobile information terminals (PDAs), portable audio players, car audio systems, game consoles, eyeglass lenses, camera lenses, lens filters, sunglasses, medical equipment (gastrocamera, etc.), copiers, personal computers (PCs), liquid-crystal displays, organic EL displays, plasma displays, touch panel displays, protective films, anti-reflection films, anti-reflection glass, templates for nanoimprint, dies, or the like.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A fluorine-containing ether compound expressed by the following formula (A1) or (A2):
{R^{f}O—(R^{f1}O)ₘ₁—(R¹)ₘ₂—(CHF)ₘ₃—O—(CHF)ₘ₄}ₙ₁-Q¹(-T¹)ₙ₂ Formula (A1)
(T²)ₙ₃-Q²-(CHF)ₘ₅—O—(CHF)ₘ₆—(R²)ₘ₇—O—(R^{f2}O)ₘ₈—(R³)ₘ₉—(CHF)ₘ₁₀—O—(CHF)ₘ₁₁-Q³(-T³)ₙ₄ Formula (A2),
wherein
R^{f} is a fluoroalkyl group having a carbon number of from 1 to 20, and when there are a plurality of R^{f}, the plurality of R^{f} may be identical to or different from each other,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom, and when there are a plurality of R¹, the plurality of R¹ may be identical to or different from each other,
Q¹ is an (n1+n2)-valent linking group,
Q² is a (1+n3)-valent linking group,
Q³ is a (1+n4)-valent linking group,
T¹, T², and T³ are each independently an adhesive group, and when there are a plurality of T¹, a plurality of T², or a plurality of T³, the plurality of T¹, the plurality of T², or the plurality of T³ may each be identical to or different from each other,
m1 and m8 are each independently an integer of from 0 to 210, and when there are a plurality of m1, the plurality of m1 may be identical to or different from each other,
m2, m7, and m9 are each independently 0 or 1, and when there are a plurality of m2, the plurality of m2 may be identical to or different from each other,
m3 and m4 are each independently 0 or 1, m3+m4 is 1 or 2, and when there are a plurality of (CHF)ₘ₃—O—(CHF)ₘ₄, the combinations of m3 and m4 may be identical to or different from each other,
m1+m2 is 1 or greater,
m7+m8+m9 is 1 or greater,
m5 and m6 are each independently 0 or 1, and m5+m6 is 1 or 2,
m10 and m11 are each independently 0 or 1, and m10+m11 is 1 or 2,
n1 is an integer of from 1 to 10, and
n2, n3, and n4 are each independently an integer of from 1 to 20.

2. The fluorine-containing ether compound according to claim 1, wherein the m3, m6, or m10 is 1.

3. The fluorine-containing ether compound according to claim 1, wherein the m4, m5, or m11 is 0.

4. The fluorine-containing ether compound according to claim 2, wherein the m4, m5, or m11 is 0.

5. A compound expressed by the following formula (B1) or (B2):
R^{f}O—(R^{f1}O)ₘ₁—(R¹)ₘ₂—CHO Formula (B1)
OHC—(R²)ₘ₇—O—(R^{f2}O)ₘ₈—(R³)ₘ₉—CHO Formula (B2),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
m1 and m8 are each independently an integer of from 0 to 210, and
m2, m7, and m9 are each independently 0 or 1.

6. A method of manufacturing a fluorine-containing ether compound, the method comprising:
reacting a compound expressed by the following formula (B1) or (B2) and a compound expressed by the following formula (C1):
R^{f}O—(R^{f1}O)ₘ₁—(R¹)ₘ₂—CHO Formula (B1)
OHC—(R²)ₘ₇—O—(R^{f2}O)ₘ₈—(R³)ₘ₉—CHO Formula (B2)
(CH₂═CH—)ₙ₅-Q⁴(—OH)ₙ₆ Formula (C1),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
Q⁴ is an (n5+n6)-valent linking group,
m1 and m8 are each independently an integer of from 0 to 210,
m2, m7, and m9 are each independently 0 or 1,
n5 is an integer of from 1 to 20, and
n6 is an integer of from 1 to 10.

7. A method of manufacturing a compound expressed by the following formula (B1) or (B2), the method comprising:
reacting a compound expressed by the following formula (D1) or (D2) with a silicon hydride compound in the presence of a Lewis acid compound to obtain a compound expressed by the following formula (E1) or (E2);
eliminating Si(R¹⁶)₃ from the compound expressed by the following formula (E1) or (E2) to obtain a compound expressed by the following formula (F1) or (F2); and
heating the compound expressed by the following formula (F1) or (F2):
R^{f}O—(R^{f1}O)ₘ₁—(R¹)ₘ₂—C(═O)OR¹¹ Formula (D1)
R¹¹OC(═O)—(R²)ₘ₇—O—(R^{f2}O)ₘ₈—(R³)ₘ₉—C(═O)OR¹¹ Formula (D2)
R^{f}O—(R^{f1}O)ₘ₁—(R¹)ₘ₂—CH(OR¹²)₂ Formula (E1)
(R¹²O)₂CH—(R²)ₘ₇—O—(R¹²O)ₘ₈—(R³)ₘ₉—CH(OR¹³)₂ Formula (E2)
R^{f}O—(R^{f1}O)ₘ₁—(R¹)ₘ₂—CH(OR¹⁴)₂ Formula (F1)
(R¹⁴O)₂CH—(R²)ₘ₇—O—(R^{f2}O)ₘ₈—(R³)ₘ₉—CH(OR¹⁵)₂ Formula (F2)
R^{f}O—(R^{f1}O)ₘ₁—(R¹)ₘ₂—CHO Formula (B1)
OHC—(R²)ₘ₇—O—(R^{f2}O)ₘ₈—(R³)ₘ₉—CHO Formula (B2),
wherein
R^{f} is a fluoroalkyl group having a carbon number of from 1 to 20,
R^{f1} and R^{f2} are each independently a fluoroalkylene group having a carbon number of from 1 to 6, and when there are a plurality of R^{f1} or a plurality of R^{f2}, the plurality of R^{f1} or the plurality of R^{f2} may each be identical to or different from each other,
R¹, R², and R³ are each independently an alkylene group that may have a fluorine atom,
R¹¹ is an alkyl group that may have a hydrogen atom or a substituent, and when there are a plurality of R¹¹, the plurality of R¹¹ may be identical to or different from each other,
R¹² and R¹³ are each independently Si(R¹⁶)₃ or R¹¹, and at least one of the plurality of R¹² and at least one of the plurality of R¹³ are Si(R¹⁶)₃,
R¹⁴ and R¹⁵ are each independently a hydrogen atom or R¹¹, and at least one of the plurality of R¹⁴ and at least one of the plurality of R¹⁵ are a hydrogen atom, and
R¹⁶ is a hydrogen atom, a halogen atom, an alkyl group, an aryl group, or an alkoxy group, and the plurality of R¹⁶ may be identical to or different from each other,
m1 and m8 are each independently an integer of from 0 to 210, and
m2, m7, and m9 are each independently 0 or 1.

8. A fluorine-containing ether composition comprising:
one or more kinds of the fluorine-containing ether compound according to claim 1; and
another fluorine-containing ether compound.

9. A coating liquid comprising:
the fluorine-containing ether compound according to claim 1, and
a liquid medium.

10. An article comprising:
a surface layer formed of the fluorine-containing ether compound according to claim 1.

11. An article comprising:
a surface layer formed of the fluorine-containing ether composition according to claim 7.

12. A method of manufacturing an article, the method comprising:
forming a surface layer through a dry coating technique or a wet coating technique by using the fluorine-containing ether compound according to claim 1.

13. A method of manufacturing an article, the method comprising:
forming a surface layer through a dry coating technique or a wet coating technique by using the fluorine-containing ether composition according to claim 7.

14. A method of manufacturing an article, the method comprising:
forming a surface layer through a dry coating technique or a wet coating technique by using the coating liquid according to claim 8.
